SEQUENCE LISTING (119 sequences)

SEQ ID NO: 1 - protein, 7 aa, synthetic construct
  source 1..7: note = anti-Claudin18.2 CDR1-H
GSIFLIN

SEQ ID NO: 2 - protein, 5 aa, synthetic construct
  source 1..5: note = anti-Claudin18.2 CDR2-H
TRGGS

SEQ ID NO: 3 - protein, 14 aa, synthetic construct
  source 1..14: note = anti-Claudin18.2 CDR3-H
DLNLRSDPFKWYTF

SEQ ID NO: 4 - protein, 122 aa, synthetic construct
  source 1..122: note = anti-Claudin18.2 VHH
QVQLVESGGGLVQPGGSLRLSCAASGSIFLINAMGWYRQAPGKQRELVAVITRGGSANYTDSVKGRFTISRDNAKNTVYLQMNSLRAEDTAVYYCNADLNLRSDPFKWYTFWGQGTLVTVSS

SEQ ID NO: 5 - protein, 7 aa, synthetic construct
  source 1..7: note = anti-CD19 CDR1-H
GVSLPDY

SEQ ID NO: 6 - protein, 5 aa, synthetic construct
  source 1..5: note = anti-CD19 CDR2-H
WGSET

SEQ ID NO: 7 - protein, 12 aa, synthetic construct
  source 1..12: note = anti-CD19 CDR3-H
HYYYGGSYAMDY

SEQ ID NO: 8 - protein, 11 aa, synthetic construct
  source 1..11: note = anti-CD19 CDR1-L
RASQDISKYLN

SEQ ID NO: 9 - protein, 7 aa, synthetic construct
  source 1..7: note = anti-CD19 CDR2-L
HTSRLHS

SEQ ID NO: 10 - protein, 9 aa, synthetic construct
  source 1..9: note = anti-CD19 CDR3-L
QQGNTLPYT

SEQ ID NO: 11 - protein, 120 aa, synthetic construct
  source 1..120: note = anti-CD19 VH
QVTLKESGPVLVKPTETLTLTCTVSGVSLPDYGVSWIRQPPGKALEWLAVIWGSETTYYNSALKSRLTISKDTSKSQVVLTMTNMDPVDTATYYCARHYYYGGSYAMDYWGQGTMVTVSS

SEQ ID NO: 12 - protein, 106 aa, synthetic construct
  source 1..106: note = anti-CD19 VL
DIQMTQSPSSLSASVGDRVTITCRASQDISKYLNWYQQKPGKAPKLLLYHTSRLHSGVPSRFSGSGSGTDYTLTISSLQQEDFATYYCQQGNTLPYTFGQGTKVEI

SEQ ID NO: 13 - protein, 241 aa, synthetic construct
  source 1..241: note = anti-CD19 scFv
DIQMTQSPSSLSASVGDRVTITCRASQDISKYLNWYQQKPGKAPKLLLYHTSRLHSGVPSRFSGSGSGTDYTLTISSLQQEDFATYYCQQGNTLPYTFGQGTKVEIGGGGSGGGGSGGGGSQVTLKESGPVLVKPTETLTLTCTVSGVSLPDYGVSWIRQPPGKALEWLAVIWGSETTYYNSALKSRLTISKDTSKSQVVLTMTNMDPVDTATYYCARHYYYGGSYAMDYWGQGTMVTVSS

SEQ ID NO: 14 - protein, 27 aa, synthetic construct
  source 1..27: note = CD28 transmembrane domain
FWVLVVVGGVLACYSLLVTVAFIIFWV

SEQ ID NO: 15 - protein, 25 aa, synthetic construct
  source 1..25: note = CD8a transmembrane domain
IYIWAPLAGTCGVLLLSLVITLYCK

SEQ ID NO: 16 - protein, 21 aa, synthetic construct
  source 1..21: note = mCD8a transmembrane domain
IWAPLAGICVALLLSLIITLI

SEQ ID NO: 17 - protein, 41 aa, synthetic construct
  source 1..41: note = CD28 costimulatory domain
RSKRSRLLHSDYMNMTPRRPGPTRKHYQPYAPPRDFAAYRS

SEQ ID NO: 18 - protein, 40 aa, synthetic construct
  source 1..40: note = 4-1BB costimulatory domain
RGRKKLLYIFKQPFMRPVQTTQEEDGCSCRFPEEEEGGCE

SEQ ID NO: 19 - protein, 42 aa, synthetic construct
  source 1..42: note = m4-1BB costimulatory domain
RKKFPHIFKQPFKKTTGAAQEEDACSCRCPQEEEGGGGGYEL

SEQ ID NO: 20 - protein, 114 aa, synthetic construct
  source 1..114: note = CD3zm intracellular domain
LRVKFSRSADAPAYQQGQNQLFNELNLGRREEFDVLDKRRGRDPEMGGKPQRRKNPQEGLYNELQKDKMAEAYSEIGMKGERRRGKGHDGLFQGLSTATKDTFDALHMQALPPR

SEQ ID NO: 21 - protein, 113 aa, synthetic construct
  source 1..113: note = CD3z intracellular domain
LRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 109 aa, synthetic construct
  source 1..109: note = mCD3z intracellular domain
SRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 23 - protein, 20 aa, synthetic construct
  source 1..20: note = B2M signal peptide
MSRSVALAVLALLSLSGLEA

SEQ ID NO: 24 - protein, 21 aa, synthetic construct
  source 1..21: note = CD8a signal peptide
MALPVTALLLPLALLLHAARP

SEQ ID NO: 25 - protein, 24 aa, synthetic construct
  source 1..24: note = mCD8a signal peptide
MASPLTRFLSLNLLLLGESIILGS

SEQ ID NO: 26 - protein, 39 aa, synthetic construct
  source 1..39: note = CD28 hinge
IEVMYPPPYLDNEKSNGTIIHVKGKHLCPSPLFPGPSKP

SEQ ID NO: 27 - protein, 45 aa, synthetic construct
  source 1..45: note = CD8a hinge
TTTPAPRPPTPAPTIASQPLSLRPEACRPAAGGAVHTRGLDFACD

SEQ ID NO: 28 - protein, 45 aa, synthetic construct
  source 1..45: note = mCD8a hinge
TTTKPVLRTPSPVHPTGTSQPQRPEDCRPRGSVKGTGLDFACDIY

SEQ ID NO: 29 - protein, 12 aa, synthetic construct
  source 1..12: note = IgG4 hinge
ESKYGPPCPPCP

SEQ ID NO: 30 - protein, 18 aa, synthetic construct
  source 1..18: note = T2A
EGRGSLLTCGDVEENPGP

SEQ ID NO: 31 - protein, 15 aa, synthetic construct
  source 1..15: note = linker
GGGGSGGGGSGGGGS

SEQ ID NO: 32 - protein, 18 aa, synthetic construct
  source 1..18: note = linker
GSTSGSGKPGSGEGSTKG

SEQ ID NO: 33 - protein, 8 aa, synthetic construct
  source 1..8: note = anti-ROR1 CDR1-H
AYSITSDY

SEQ ID NO: 34 - protein, 5 aa, synthetic construct
  source 1..5: note = anti-ROR1 CDR2-H
SYSGF

SEQ ID NO: 35 - protein, 9 aa, synthetic construct
  source 1..9: note = anti-ROR1 CDR3-H
GEITYYFDY

SEQ ID NO: 36 - protein, 11 aa, synthetic construct
  source 1..11: note = anti-ROR1 CDR1-L
ITSTDIDEDMN

SEQ ID NO: 37 - protein, 7 aa, synthetic construct
  source 1..7: note = anti-ROR1 CDR2-L
EGNTLRP

SEQ ID NO: 38 - protein, 9 aa, synthetic construct
  source 1..9: note = anti-ROR1 CDR3-L
LQSNNMPLT

SEQ ID NO: 39 - protein, 118 aa, synthetic construct
  source 1..118: note = anti-ROR1 VH
QVQLQESGPGLVKPSQTLSLTCTVTAYSITSDYAWNWIRQFPGNKLEWMGYISYSGFTRYNPSLKSRISITRDTSKNQFFLKLNFVTTEDTATYYCARGEITYYFDYWGQGTTLTVSS

SEQ ID NO: 40 - protein, 107 aa, synthetic construct
  source 1..107: note = anti-ROR1 VL
ETTLTQSPATLSVSPGERAIIRCITSTDIDEDMNWYQQKPGQPPKLLISEGNTLRPGVPSRFSGSGSGTDFTLTISSLQPEDVATYYCLQSNNMPLTFGAGTKLELK

SEQ ID NO: 41 - protein, 240 aa, synthetic construct
  source 1..240: note = anti-ROR1 scFv
ETTLTQSPATLSVSPGERAIIRCITSTDIDEDMNWYQQKPGQPPKLLISEGNTLRPGVPSRFSGSGSGTDFTLTISSLQPEDVATYYCLQSNNMPLTFGAGTKLELKGGGGSGGGGSGGGGSQVQLQESGPGLVKPSQTLSLTCTVTAYSITSDYAWNWIRQFPGNKLEWMGYISYSGFTRYNPSLKSRISITRDTSKNQFFLKLNFVTTEDTATYYCARGEITYYFDYWGQGTTLTVSS

SEQ ID NO: 42 - protein, 7 aa, synthetic construct
  source 1..7: note = anti-NKG2A CDR1-H
GFTFSSY

SEQ ID NO: 43 - protein, 6 aa, synthetic construct
  source 1..6: note = anti-NKG2A CDR2-H
SSGGSY

SEQ ID NO: 44 - protein, 10 aa, synthetic construct
  source 1..10: note = anti-NKG2A CDR3-H
HGDYPRFFDV

SEQ ID NO: 45 - protein, 11 aa, synthetic construct
  source 1..11: note = anti-NKG2A CDR1-L
SASSSVSSYIY

SEQ ID NO: 46 - protein, 7 aa, synthetic construct
  source 1..7: note = anti-NKG2A CDR2-L
LTSNLAS

SEQ ID NO: 47 - protein, 9 aa, synthetic construct
  source 1..9: note = anti-NKG2A CDR3-L
QQWSGNPYT

SEQ ID NO: 48 - protein, 119 aa, synthetic construct
  source 1..119: note = anti-NKG2A VH
EVQLVESGGGLVKPGGSLRLSCAASGFTFSSYAMSWVRQAPGKGLEWVAEISSGGSYTYYPDSVKGRFTISRDNAKNSLYLQMNSLRAEDTAVYYCTRHGDYPRFFDVWGQGTLVTVSS

SEQ ID NO: 49 - protein, 107 aa, synthetic construct
  source 1..107: note = anti-NKG2A VL
EIVLTQSPATLSLSPGERATMTCSASSSVSSYIYWYQQKPGQAPRLLIYLTSNLASGIPARFSGSGSGTDFTLTISSLEPEDFATYYCQQWSGNPYTFGQGTKVEIK

SEQ ID NO: 50 - protein, 244 aa, synthetic construct
  source 1..244: note = anti-NKG2A scFv
EIVLTQSPATLSLSPGERATMTCSASSSVSSYIYWYQQKPGQAPRLLIYLTSNLASGIPARFSGSGSGTDFTLTISSLEPEDFATYYCQQWSGNPYTFGQGTKVEIKGSTSGSGKPGSGEGSTKGEVQLVESGGGLVKPGGSLRLSCAASGFTFSSYAMSWVRQAPGKGLEWVAEISSGGSYTYYPDSVKGRFTISRDNAKNSLYLQMNSLRAEDTAVYYCTRHGDYPRFFDVWGQGTLVTVSS

SEQ ID NO: 51 - protein, 284 aa, synthetic construct
  source 1..284: note = HLA-E extracellular domain
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPI

SEQ ID NO: 52 - protein, 284 aa, synthetic construct
  source 1..284: note = HLA-E extracellular domain mutant
GSHSLKYFHTSVSRPGRGEPRFISVGYVDDTQFVRFDNDAASPRMVPRAPWMEQEGSEYWDRETRSARDTAQIFRVNLRTLRGCYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPI

SEQ ID NO: 53 - protein, 7 aa, synthetic construct
  source 1..7: note = anti-PD1 CDR1-H
GYTFTNY

SEQ ID NO: 54 - protein, 6 aa, synthetic construct
  source 1..6: note = anti-PD1 CDR2-H
NPSNGG

SEQ ID NO: 55 - protein, 11 aa, synthetic construct
  source 1..11: note = anti-PD1 CDR3-H
RDYRFDMGFDY

SEQ ID NO: 56 - protein, 15 aa, synthetic construct
  source 1..15: note = anti-PD1 CDR1-L
RASKGVSTSGYSYLH

SEQ ID NO: 57 - protein, 7 aa, synthetic construct
  source 1..7: note = anti-PD1 CDR2-L
LASYLES

SEQ ID NO: 58 - protein, 9 aa, synthetic construct
  source 1..9: note = anti-PD1 CDR3-L
QHSRDLPLT

SEQ ID NO: 59 - protein, 120 aa, synthetic construct [sequence omitted]

SEQ ID NO: 60 - protein, 111 aa, synthetic construct [sequence omitted]

SEQ ID NO: 61 - protein, 246 aa, synthetic construct
  source 1..246: note = anti-PD1 scFv
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK

SEQ ID NO: 62 - protein, 18 aa, synthetic construct
  source 1..18: note = PDL1 signal peptide
MRIFAVFIFMTYWHLLNA

SEQ ID NO: 63 - protein, 20 aa, synthetic construct
  source 1..20: note = PDL1 transmembrane domain
VILGAILLCLGVALTFIFRL

SEQ ID NO: 64 - protein, 223 aa, synthetic construct
  source 1..223: note = PDL1 extracellular domain
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHL

SEQ ID NO: 65 - protein, 19 aa, synthetic construct
  source 1..19: note = PDL2 signal peptide
MIFLLLMLSLELQLHQIAA

SEQ ID NO: 66 - protein, 21 aa, synthetic construct
  source 1..21: note = PDL2 transmembrane domain
WLLHIFIPSCIIAFIFIATVI

SEQ ID NO: 67 - protein, 201 aa, synthetic construct
  source 1..201: note = PDL2 extracellular domain
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPT

SEQ ID NO: 68 - protein, 7 aa, synthetic construct
  source 1..7: note = anti-FasL CDR1-H
GYIFISH

SEQ ID NO: 69 - protein, 6 aa, synthetic construct
  source 1..6: note = anti-FasL CDR2-H
NAYSGN

SEQ ID NO: 70 - protein, 11 aa, synthetic construct
  source 1..11: note = anti-FasL CDR3-H
ETMVRGVPCDY

SEQ ID NO: 71 - protein, 12 aa, synthetic construct
  source 1..12: note = anti-FasL CDR1-L
RASQSVSSSYLA

SEQ ID NO: 72 - protein, 7 aa, synthetic construct
  source 1..7: note = anti-FasL CDR2-L
GASSRAT

SEQ ID NO: 73 - protein, 9 aa, synthetic construct
  source 1..9: note = anti-FasL CDR3-L
QQYGSSPWT

SEQ ID NO: 74 - protein, 120 aa, synthetic construct
  source 1..120: note = anti-FasL VH
QVQLVQSGAEVKKPGASVKVSCKASGYIFISHGISWVRQAPGQGLEWMGWINAYSGNTNYAQKLQGRVTMTTDRSTSTAYMELRSLRSDDTAVYYCARETMVRGVPCDYWGQGTLVTVSS

SEQ ID NO: 75 - protein, 109 aa, synthetic construct
  source 1..109: note = anti-FasL VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKR

SEQ ID NO: 76 - protein, 244 aa, synthetic construct
  source 1..244: note = anti-FasL scFv
QVQLVQSGAEVKKPGASVKVSCKASGYIFISHGISWVRQAPGQGLEWMGWINAYSGNTNYAQKLQGRVTMTTDRSTSTAYMELRSLRSDDTAVYYCARETMVRGVPCDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKR

SEQ ID NO: 77 - protein, 173 aa, synthetic construct
  source 1..173: note = Fas extracellular domain
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSN

SEQ ID NO: 78 - protein, 17 aa, synthetic construct
  source 1..17: note = Fas transmembrane domain
LGWLCLLLLPIPLIVWV

SEQ ID NO: 79 - protein, 7 aa, synthetic construct
  source 1..7: note = anti-CTLA4 CDR1-H
GFTFSSY

SEQ ID NO: 80 - protein, 6 aa, synthetic construct
  source 1..6: note = anti-CTLA4 CDR2-H
WYDGSN

SEQ ID NO: 81 - protein, 10 aa, synthetic construct
  source 1..10: note = anti-CTLA4 CDR3-H
APNYIGAFDV

SEQ ID NO: 82 - protein, 11 aa, synthetic construct
  source 1..11: note = anti-CTLA4 CDR1-L
RASQGISSWLA

SEQ ID NO: 83 - protein, 7 aa, synthetic construct
  source 1..7: note = anti-CTLA4 CDR2-L
AASSLQS

SEQ ID NO: 84 - protein, 9 aa, synthetic construct
  source 1..9: note = anti-CTLA4 CDR3-L
QQYNSYPPT

SEQ ID NO: 85 - protein, 119 aa, synthetic construct
  source 1..119: note = anti-CTLA4 VH
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVFYCARAPNYIGAFDVWGQGTMVTVSS

SEQ ID NO: 86 - protein, 107 aa, synthetic construct
  source 1..107: note = anti-CTLA4 VL
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGQGTKVEIK

SEQ ID NO: 87 - protein, 241 aa, synthetic construct
  source 1..241: note = anti-CTLA4 scFv
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVFYCARAPNYIGAFDVWGQGTMVTVSSGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGQGTKVEIK

SEQ ID NO: 88 - protein, 246 aa, synthetic construct
  source 1..246: note = CD80 extracellular domain
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPS

SEQ ID NO: 89 - protein, 232 aa, synthetic construct
  source 1..232: note = CD86 extracellular domain
FVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDH

SEQ ID NO: 90 - protein, 22 aa, synthetic construct
  source 1..22: note = CD80 transmembrane domain
WAITLISVNGIFVICCLTYCFA

SEQ ID NO: 91 - protein, 23 aa, synthetic construct
  source 1..23: note = CD86 transmembrane domain
IPWITAVLPTVIICVMVFCLILW

SEQ ID NO: 92 - protein, 7 aa, synthetic construct
  source 1..7: note = anti-SIRPa CDR1-H
GFNISYY

SEQ ID NO: 93 - protein, 6 aa, synthetic construct
  source 1..6: note = anti-SIRPa CDR2-H
YSSFGY

SEQ ID NO: 94 - protein, 20 aa, synthetic construct
  source 1..20: note = anti-SIRPa CDR3-H
FTFPGLFTGFFGAYLGSLDY

SEQ ID NO: 95 - protein, 11 aa, synthetic construct
  source 1..11: note = anti-SIRPa CDR1-L
RASQSVSSAVA

SEQ ID NO: 96 - protein, 7 aa, synthetic construct
  source 1..7: note = anti-SIRPa CDR2-L
SASSLYS

SEQ ID NO: 97 - protein, 12 aa, synthetic construct
  source 1..12: note = anti-SIRPa CDR3-L
QQAVNWVGALVT

SEQ ID NO: 98 - protein, 129 aa, synthetic construct
  source 1..129: note = anti-SIRPa VH
EVQLVESGGGLVQPGGSLRLSCAASGFNISYYFIHWVRQAPGKGLEWVASVYSSFGYTYYADSVKGRFTISADTSKNTAYLQMNSLRAEDTAVYYCARFTFPGLFTGFFGAYLGSLDYWGQGTLVTVSS

SEQ ID NO: 99 - protein, 111 aa, synthetic construct
  source 1..111: note = anti-SIRPa VL
DIQMTQSPSSLSASVGDRVTITCRASQSVSSAVAWYQQKPGKAPKLLIYSASSLYSGVPSRFSGSRSGTDFTLTISSLQPEDFATYYCQQAVNWVGALVTFGQGTKVEIKR

SEQ ID NO: 100 - protein, 252 aa, synthetic construct
  source 1..252: note = anti-SIRPa scFv
EVQLVESGGGLVQPGGSLRLSCAASGFNISYYFIHWVRQAPGKGLEWVASVYSSFGYTYYADSVKGRFTISADTSKNTAYLQMNSLRAEDTAVYYCARFTFPGLFTGFFGAYLGSLDYWGQGTLVTVSSGGGSGGGSGGGSDIQMTQSPSSLSASVGDRVTITCRASQSVSSAVAWYQQKPGKAPKLLIYSASSLYSGVPSRFSGSRSGTDFTLTISSLQPEDFATYYCQQAVNWVGALVTFGQGTKVEIKR

SEQ ID NO: 101 - protein, 7 aa, synthetic construct
  source 1..7: note = anti-IRP60 CDR1-H
GYTFTSY

SEQ ID NO: 102 - protein, 6 aa, synthetic construct
  source 1..6: note = anti-IRP60 CDR2-H
DPSDSY

SEQ ID NO: 103 - protein, 15 aa, synthetic construct
  source 1..15: note = anti-IRP60 CDR3-H
WGMAYGTSSYWYFDV

SEQ ID NO: 104 - protein, 11 aa, synthetic construct
  source 1..11: note = anti-IRP60 CDR1-L
RASQDISNYLN

SEQ ID NO: 105 - protein, 7 aa, synthetic construct
  source 1..7: note = anti-IRP60 CDR2-L
YTSRLHS

SEQ ID NO: 106 - protein, 9 aa, synthetic construct
  source 1..9: note = anti-IRP60 CDR3-L
QQGNTLPWT

SEQ ID NO: 107 - protein, 125 aa, synthetic construct
  source 1..125: note = anti-IRP60 VH
EVQLQQSGAELVKPGASVKLSCKASGYTFTSYWMQWVKQRPGQGLEWIGEIDPSDSYTNYNQKFKGKATLTVDTSSSTTYMQLSSLTSEDSAVYYCARWGMAYGTSSYWYFDVWGTGTTVTVSSA

SEQ ID NO: 108 - protein, 107 aa, synthetic construct
  source 1..107: note = anti-IRP60 VL
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIK

SEQ ID NO: 109 - protein, 244 aa, synthetic construct
  source 1..244: note = anti-IRP60 scFv
EVQLQQSGAELVKPGASVKLSCKASGYTFTSYWMQWVKQRPGQGLEWIGEIDPSDSYTNYNQKFKGKATLTVDTSSSTTYMQLSSLTSEDSAVYYCARWGMAYGTSSYWYFDVWGTGTTVTVSSAGGGSGGGSGGGSDIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIK

SEQ ID NO: 110 - protein, 123 aa, synthetic construct
  source 1..123: note = CD47 extracellular domain-transmembrane domain
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNE

SEQ ID NO: 111 - protein, 144 aa, synthetic construct
  source 1..144: note = CD47 extracellular domain-transmembrane domain
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGI

SEQ ID NO: 112 - protein, 158 aa, synthetic construct
  source 1..158: note = CD47 extracellular domain-transmembrane domain
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKT

SEQ ID NO: 113 - protein, 179 aa, synthetic construct
  source 1..179: note = CD47 extracellular domain-transmembrane domain
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILF

SEQ ID NO: 114 - protein, 189 aa, synthetic construct
  source 1..189: note = CD47 extracellular domain-transmembrane domain
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNA

SEQ ID NO: 115 - protein, 210 aa, synthetic construct
  source 1..210: note = CD47 extracellular domain-transmembrane domain
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVF

SEQ ID NO: 116 - protein, 217 aa, synthetic construct
  source 1..217: note = CD47 extracellular domain-transmembrane domain
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLT

SEQ ID NO: 117 - protein, 238 aa, synthetic construct
  source 1..238: note = CD47 extracellular domain-transmembrane domain
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGL

SEQ ID NO: 118 - protein, 250 aa, synthetic construct
  source 1..250: note = CD47 extracellular domain-transmembrane domain
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHG

SEQ ID NO: 119 - protein, 271 aa, synthetic construct
  source 1..271: note = CD47 extracellular domain-transmembrane domain
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYM

(19) United States
(12) Patent Application Publication
(10) Pub. No.: US 2026/0365977 A1
(43) Pub. Date: Nov. 19, 2026

(54) FUNCTIONALITY-ENHANCED CELL THERAPY

(71) Applicant: IMVIVABIO LLC, BOSTON, MA (US)

(72) Inventors: Yali ZHOU, Nanjing (CN); Yingxiu TANG, Nanjing (CN); Jianqiang HOU, Nanjing (CN); Dong ZHENG, Nanjing (CN); Jiangtao REN, Nanjing (CN)

(73) Assignee: IMVIVABIO LLC, BOSTON, MA (US)

(21) Appl. No.: 19/155,237

(22) PCT Filed: Feb. 9, 2024
(86) PCT No.: PCT/CN2024/076723

§ 371 Date: Aug. 8, 2025

(30) Foreign Application Priority Data
Feb. 9, 2023 (CN) 202310091988.6

(51) Int. Cl. C12N 5/0783; A61K 35/17; A61K 40/11; A61K 40/31; A61K 40/42; A61P 35/00; C07K 14/705; C07K 16/28

(52) U.S. Cl. CPC C12N 5/0636; A61K 35/17; A61K 40/11; A61K 40/31; A61K 40/4211; A61P 35/00; C07K 14/7051; C07K 14/70539; C07K 16/2803; A61K 2239/13; C12N 2510/00

(57) ABSTRACT

The present disclosure relates to an engineered cell, wherein (i) expression of at least one of the endogenous HLA-II-related genes is down-regulated; and (ii) expression of endogenous HLA-I-related genes is not altered. The present disclosure also relates to a composition comprising the cell.

### CROSS-REFERENCE TO RELATED APPLICATION

The present disclosure claims the priority to the Chinese patent application with the application No. CN2023100919886 and entitled “FUNCTIONALITY-ENHANCED CELL THERAPY”, filed with China National Intellectual Patent Administration on Feb. 9, 2023, which is incorporated in the present disclosure by reference in its entirety.

### TECHNICAL FIELD

The present disclosure pertains to the field of immunotherapy. Specifically, the present disclosure relates to an engineered cell that reduces the risk of rejection by down-regulating the expression of endogenous HLA-II-related genes while retaining the expression of endogenous HLA-I-related genes and a composition comprising the cell.

### BACKGROUND ART

As an emerging immunotherapy, adoptive cell therapy has developed very rapidly in recent years. Particularly for chimeric antigen receptor therapy, many products have been launched and have shown excellent therapeutic effects in clinical practice. However, the products currently on the market are all of autologous therapies, which have disadvantages such as long preparation times, high costs, and high preparation failure rates. In this regard, allogeneic cell therapies have been developed to improve patient accessibility. However, allogeneic cell therapies also face a series of challenges, for example, risk of immune rejection, poor cell persistence, etc. Therefore, there is still a need to improve the existing allogeneic cell therapies to reduce risk of immune rejection and enhance cell persistence.

### SUMMARY

Unless otherwise specified, all scientific and technical terms used herein have the same meaning as commonly understood by one of ordinary skill in the art to which the present disclosure pertains.

In a first aspect, the present disclosure provides an engineered cell, wherein (i) expression of at least one of the endogenous HLA-II-related genes is down-regulated; and (ii) expression of endogenous HLA-I-related genes is not altered.

### I. Expression of Endogenous Genes

### A. Expression of Endogenous HLA Genes

The major histocompatibility complex (MHC) is a collective name for a cluster of closely linked and highly diverse genes in the vertebrate genome, which encode cell surface proteins that are essential for the immune system. Since the human MHC genes were first discovered in leukocytes, they are therefore also called human leukocyte antigens (HLA). HLA is by far the most polymorphic region in human chromosomes and is also the primary mechanism for the immune system to distinguish “self” and “non-self”. Clinically, HLA is the main immunological barrier that affects the survival time of organ transplantation or cell transplantation. Mismatch HLA between the recipient and the donor will trigger immune rejection reaction mediated by cells and antibodies. HLA is mainly classified into class I and class II.

As used herein, the term “HLA-I-related gene” refers to a gene that involves in regulating the expression of the HLA-I complex. The HLA-I complex is composed of an α chain encoded by HLA class I genes and a β chain encoded by the B2M gene via non-covalent bonds. The HLA-I complex is expressed in most types of cells in the body, and is capable of presenting endogenous antigen peptides (mostly 8-12 amino acids in length) to CD8+ T cells and inducing the killing function thereof. Wherein, the α chain includes three moieties: an extracellular region (including three domains of α1, α2, and α3), a transmembrane domain, and a cytoplasmic region, wherein the α1 and α2 domains form an antigen binding groove, which is responsible for binding to the antigen peptide entering the groove; α3 is homologous to the constant region domain of immunoglobulins and binds to the T cell surface molecule CD8. HLA class I genes mainly include HLA-A, HLA-B, and HLA-C. In the engineered cell of the present disclosure, the expression of endogenous HLA-I-related genes is not altered. In other words, the expression level of any one of the endogenous HLA-I-related genes in a cell is not altered (e.g., suppressed, knocked down, knocked out, or silenced) by any gene editing or other non-gene editing means. In an embodiment, the endogenous HLA-I-related gene refers to HLA-A, HLA-B, HLA-C, and B2M.

As used herein, the term “HLA-II-related gene” includes HLA class II genes themselves and genes that participate in or regulate the expression of HLA class II genes. HLA class II genes are mainly expressed in immune cells such as macrophages, dendritic cells, activated T cells, B cells, etc., and are capable of presenting exogenous antigen peptides (mostly 13-28 amino acids in length) to CD4+ T cells and inducing the function thereof. HLA class II molecule is composed of an α chain and a β chain, each chain containing three moieties: an extracellular region, a transmembrane domain, and a cytoplasmic region, wherein the extracellular region of both contains two structural domains. The α1 and β1 domains form an antigen binding groove, which is responsible for binding to the antigen peptide entering the groove, and the β2 domain binds to the T cell surface molecule CD4. HLA class II genes mainly include HLA-DP, HLA-DQ, and HLA-DR. In addition, there are some genes that regulate the expression of HLA class II genes by affecting the processing and transport of antigens, and such genes include, for example, LMP2, LMP7, TAP1, TAP2, HLA-DM, HLA-DN, and HLA-DO. An additional number of genes regulate the expression of HLA class II genes by binding the transcription factors of HLA-II, and such genes include, for example, RFX5, RFXAP, RFXANK, and CIITA. In the engineered cell of the present disclosure, the expression of at least one of the endogenous HLA-II-related genes is down-regulated. In an embodiment, the HLA-II-related gene is selected from the group consisting of: HLA-DP, HLA-DQ, HLA-DR, LMP2, LMP7, TAP1, TAP2, HLA-DM, HLA-DN, HLA-DO, RFX5, RFXAP, RFXANK and CIITA.

Since HLA-I- and HLA-II-related genes will activate T cells after they are recognized by the T cell receptor (TCR), and in turn induce the killing activity of T cells, and therefore, down-regulating the expression level of such genes, particularly HLA-I-related genes, is particularly important for reducing immune rejection, because CD8+ T cells are the main killer T cells subset. In fact, knocking out HLA-I-related genes has become a common practice currently in the preparation of allogeneic cell therapy. However, the inventors unexpectedly found that compared to combined knockout of HLA-I-related genes and HLA-II-related genes, knockout of HLA-II-related genes alone (i.e., retaining the expression of HLA-I-related genes) can achieve a more significant effect of reducing immune rejection. In addition, current research on HLA mostly focuses on its role in immune rejection. The inventors of the present disclosure unexpectedly found that knockout of HLA-II-related genes (i.e., retaining the expression of HLA-I-related genes) can also improve the proliferation ability of CAR-T cells, which in turn enhances the tumor suppression effect thereof.

As used herein, gene expression being “down-regulated” refers to that the expression level of the product of the modified gene is significantly reduced or almost not expressed relative to the expression level of the unmodified wild-type gene. This means that for a gene whose expression is “down-regulated”, the content of the expression product thereof (such as mRNA or protein) is reduced or the expression product lacks functionality. Therefore, down-regulation can be achieved in different dimensions such as gene level, transcription level, and translation level. Generally, gene expression is down-regulated by methods well known to those skilled in the art, including but not limited to, for example, knocking down or knocking out a gene of interest itself or the regulatory gene of the gene of interest by technologies such as meganucleases, zinc finger nucleases, TALENs, CRISPR/Cas systems, base editors, and lead editors, or reducing the expression level of the gene of interest itself or the regulatory gene of the gene of interest by technologies such as antisense oligonucleotides, RNAi, shRNAs, transposons, siRNAs, antigomer RNAs, mutagenesis, antibodies, or chemical inhibitors.

### B. Expression of Other Endogenous Genes

In an embodiment, the engineered cell of the present disclosure further comprises down-regulation of expression levels of other endogenous genes to further reduce the risk of cell rejection, or enhance the efficacy of the cells, for example, improving the amplification activity/killing activity of the cells, prolonging the persistence of the cells, and the like.

In an embodiment, wherein the expression of at least one one of the endogenous TCR/CD3 genes of the engineered cell is down-regulated. The T cell surface receptor (TCR) is a characteristic marker on the surface of all T cells, which binds to CD3 with a non-covalent bond to form a TCR/CD3 complex, and generates a specific antigen stimulation signal by binding to the specific MHC-antigen peptide complex on the surface of the antigen-presenting cell to activate the T cell and play a killing effect. Therefore, suppressing or silencing the expression of the endogenous TCR/CD3 gene in CAR-T cells is capable of avoiding its attack on normal cells or tissues in a patient, which in turn avoids or reduces the risk of graft-versus-host disease (GvHD). In an embodiment, the TCR/CD3 gene is selected from the group consisting of: TRAC, TRBC, CD3γ, CD3δ, CD3ε, and Cd3ζ.

In an embodiment, the expression of at least one of the endogenous gene of the engineered cell is down-regulated, the at least one of the endogenous gene selected from the group consisting of: CD52, GR, dCK, PD1, LAG3, TIM3, CTLA4, PPP2CA, PPP2CB, PTPN6, PTPN22, PDCD1, HAVCR2, BTLA, CD160, TIGIT, CD96, CRTAM, TNFR1, DR3, DR4, DR5, TNFRSF10B, TNFRSF10A, CASP8, CASP10, CASP3, CASP6, CASP7, FADD, FAS, TGFBR1, TGFBR2, SMAD2, SMAD3, SMAD4, SMAD10, SKI, SKIL, TGIF1, IL10RA, IL10RB, HMOX2, IL6R, IL6ST, EIF2AK4, CSK, PAG1, SIT, FOXP3, PRDM1, BATF, GUCY1A2, GUCY1A3, GUCY1B2 and GUCY1B3, more preferably selected from the group consisting of: FAS, TGFBR1, TGFBR2, TNFR1, DR3, DR4 and DR5.

Fas, TNFR1, DR3, DR4 and DR5 all pertain to the death receptor (DR) family, and share a Cys-rich extracellular domain and an intracellular death domain (DD). When these death receptors bind to their specific death ligands, they receive extracellular death signals, activate the intracellular apoptotic mechanism, and induce apoptosis. TGFBR1 and TGFBR2 are both receptors for TGFB1, and both contain serine/threonine kinase activity in the intracellular regions. They mediate Smad-dependent and -independent signaling pathways, and play an important regulatory role in processes such as cell proliferation, differentiation, wound healing, and tumorigenesis and metastasis. The inventors found that down-regulating the expression of these endogenous genes in cells is capable of significantly prolonging cell persistence and improving cell survival rate.

### II. Immunosuppressive Molecules (IIM)

The engineered cell provided in the present disclosure also expresses an exogenous immunosuppressive molecule, wherein the immunosuppressive is capable of reducing the immunogenicity of the engineered cell.

As used herein, the term “immunosuppressive molecule” refers to a molecule capable of binding to an immunosuppressive protein and in turn to suppress immune rejection of exogenous cells by a subject, for example, reducing the killing function of immune cells (e.g., T cells and NK cells) in the subject or suppressing the excessive proliferation of immune cells. In an embodiment, the immunosuppressive molecule comprises one or more immunosuppressive protein-binding domains, a transmembrane domain, and a co-stimulatory domain and does not comprise a primary signaling domain.

As used herein, the term “immunosuppressive protein-binding domain” refers to a domain capable of specifically binding to an immunosuppressive protein. In an embodiment, the immunosuppressive protein-binding domain is an antibody targeting an immunosuppressive protein, or an extracellular region of a ligand of an immunosuppressive protein. In an embodiment, such immunosuppressive proteins include, but are not limited to: a NKG2/CD94 component (e.g., NKG2A, NKG2B, CD94); a member of the killer cell Ig-like receptor (KIR) family (e.g., KIR2DL1, KIR2DL2/3, KIR2DL5A, KIR2DL5B, KIR3DL1, KIR3DL2, and KIR3DL3); a member of the leukocyte Ig-like receptor (LIR) family (e.g., LIR1, LIR2, LIR3, LIR5, and LIR8); a member of the NK cell receptor protein 1 (NKR-P1) family (e.g., NKR-P1B and NKR-P1D); an immune checkpoint receptor (e.g., PD-1, TIGIT, CD96, TIM3, LAG3 and CTLA4); carcinoembryonic antigen-related cell adhesion molecule 1 (CEACAM1); a member of the sialic acid-binding immunoglobulin-like lectin (SIGLEC) family (e.g., SIGLEC7 and SIGLEC9); leukocyte-associated immunoglobulin-like receptor 1 (LAIR1); a member of the Ly49 family (e.g., Ly49A, Ly49C, Ly49F, Ly49G1 and Ly49G4), killer cell lectin-like receptor G1 (KLRG1), and FASL. Preferably, the immunosuppressive protein is selected from the group consisting of: PD1, TIGIT, CD96, TIM3, LAG3, CTLA4, FASL, NKG2A, NKG2B, CD94, LIR1, LIR2, LIR3, LIR5, LIR8, KIR2DL1, KIR2DL2/3, KIR3DL1, CEACAM1, LAIR1, SIGLEC7, SIGLEC9, SIRP-α, IRp60 and KLRG1.

In an embodiment, the immunosuppressive protein-binding domain is an antibody targeting an immunosuppressive protein, for example, an antibody that specifically binds to the following targets: PD1, TIGIT, CD96, TIM3, LAG3, CTLA4, FASL, NKG2A, NKG2B, CD94, LIR1, LIR2, LIR3, LIR5, LIR8, KIR2DL1, KIR2DL2/3, KIR3DL1, CEACAM1, LAIR1, SIGLEC7, SIGLEC9, SIRP-α, IRp60, and KLRG1. Known antibodies to the above targets can all be used in the present disclosure. In an embodiment, the immunosuppressive protein-binding domain is an extracellular region of an immunosuppressive protein ligand, for example, an extracellular region of a protein selected from the group consisting of: HLA-E, HLA-F, HLA-G, cadherin, collagen, OCIL, sialic acid, PDL1, PDL2, CD155, CD112, CD113, Gal-9, FGL1, FAS, CD80, CD86, CD47.

As used herein, the term “antibody” has the broadest meaning understood by those skilled in the art and includes monoclonal antibodies (comprising whole antibodies), polyclonal antibodies, multivalent antibodies, multispecific antibodies (e.g. bispecific antibodies), and antibody fragments or synthetic polypeptides carrying one or more CDR sequences capable of exhibiting the desired biological activity. The antibody of the present disclosure may be of any class (e.g. IgG, IgE, IgM, IgD, IgA, etc.) or subclass (e.g. IgG1, IgG2, IgG2a, IgG3, IgG4, IgA1, IgA2, etc.). As used herein, the term “antibody fragment” refers to one or more fragments of an antibody that retain the ability to specifically bind an antigen. It has been shown that the antigen binding function of an antibody can be performed by the fragment of a full-length antibody. Examples of antibody fragment of the present disclosure include, but are not limited to: Fab, Fab′, F(ab′)2, Fd fragment, Fd′, Fv fragment, single-chain antibody (scFv), disulfide-linked Fv (sdFv), antibody heavy chain variable region (VH) or light chain variable region (VL), “diabody” with two antigen binding sites, single domain antibody (sdAb), nanobody, etc. Therefore, unless the context clearly dictates otherwise, the “antibody” of the present disclosure encompasses antibody fragments as defined above. Therefore, in a preferred embodiment, the antibody of the present disclosure is selected from the group consisting of IgG, Fab, Fab′, F(ab′)₂, Fd, Fd′, Fv, scFv, sdFv, diabody, sdAb or nanobody, preferably scFv, sdAb or nanobody. In an embodiment, the antibody of the present disclosure is a murine antibody, a chimeric antibody, a camel-derived antibody, a humanized antibody or a human antibody.

Typically, an intact antibody includes two heavy chains and two light chains disulfide-linked together, each light chain being disulfide-linked to a respective heavy chain, to form a “Y” configuration. Each heavy chain comprises a heavy chain variable region (VH) and a heavy chain constant region, wherein the heavy chain variable region comprises three complementarity determining regions (CDRs): CDR1-H, CDR2-H and CDR3-H, and the heavy chain constant region comprises three constant domains: CH1, CH2 and CH3. Each light chain comprises a light chain variable region (VL) and a light chain constant region, wherein the light chain variable region comprises three CDRs: CDR1-L, CDR2-L and CDR3-L, and the light chain constant region comprises a constant domain CL. In the heavy/light chain variable regions, the CDRs are separated by more conserved framework regions (FRs). The heavy/light chain variable regions are responsible for the recognition and binding to the antigen, while the constant regions can mediate the binding of the antibody to host tissues or factors, including various cells of the immune system (e.g., effector cells) and the first component of the classical complement system.

The precise amino acid sequence boundaries for a given CDR or FR can be readily determined using a number of numbering schemes well known in the art, these schemes including: Kabat et al. (1991), “Sequences of Proteins of Immunological Interest,” 5th Edition, Public Health Service, National Institutes of Health, Bethesda, MD (“Kabat” numbering scheme); Al-Lazikani et al., (1997) JMB 273, 927-948 (“Chothia” numbering scheme); MacCallum et al., J. Mol. Biol 262:732-745 (1996), “Antibody-antigen interactions: Contact analysis and binding sitetopography,” J. Mol. Biol. 262, 732-745″ (“Contact” numbering scheme); Lefranc M P et al., “IMGT unique numbering for immunoglobulin and T cell receptor variable domains and Ig superfamily V-like domains,” Dev Comp Immunol, 2003 January; 27 (1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309 (3): 657-70 (“Aho” numbering scheme); and Martin et al., “Modeling antibody hypervariable loops: a combined algorithm,” PNAS, 1989, 86 (23): 9268-9272 (“AbM” numbering scheme).

The boundaries of a given CDR or FR may vary depending on the scheme used for definition. For example, the Kabat scheme is based on structural alignment, while the Chothia scheme is based on structural information. Both the Kabat and Chothia numbering schemes are based on the sequence length of the most common antibody regions wherein insertions are provided by caret letters (e.g. “30a”) and deletions occur in some antibodies. These two schemes place certain insertions and deletions (indels) at different positions, thereby resulting in different numbering. The Contact scheme is based on the analysis of complex crystal structures and is similar in many respects to the Chothia numbering scheme. The AbM scheme is a compromise between the Kabat and Chothia definitions and is based on the scheme used by the AbM antibody modeling software of Oxford Molecular.

Therefore, unless otherwise specified, a “CDR” of a given antibody or region thereof (e.g. variable region thereof) is understood to encompass the CDRs as defined by any of the above schemes or other known schemes. For example, where it is specified that a particular CDR (e.g. CDR3) comprises a given amino acid sequence, it is understood that such a CDR may further have the sequence of the corresponding CDR (e.g. CDR3) as defined by any of the above schemes or other known schemes. Likewise, unless otherwise specified, an FR for a given antibody or region thereof (e.g. variable region thereof) is understood to encompass the FRs as defined by any of the above schemes or other known schemes. Unless otherwise specified, the numbering scheme used herein to define the boundaries of CDRs and FRs adopts the Chothia scheme.

As used herein, the term sequence “identity” means the degree to which two (nucleotide or amino acid) sequences in alignment have the same residue at the same position, and is usually expressed as a percentage. Preferably, identity is determined over the entire length of the sequences being compared. Therefore, two copies of the exact same sequence have 100% identity. Those skilled in the art know that several algorithms can be used to determine sequence identity, for example, Blast (Altschul et al. (1997) Nucleic Acids Res. 25:3389-3402), Blast2 (Altschul et al. (1990) J. Mol. Biol. 215:403-410), Smith-Waterman (Smith et al. (1981) J. Mol. Biol. 147:195-197) and Clustal W.

In an embodiment, the immunosuppressive protein-binding domain of the present disclosure is a PD1-targeting antibody.

Anti-PD1 antibodies known in the art can all be used in the present disclosure. In an embodiment, the PD1-targeting antibody comprises a light chain variable region and a heavy chain variable region, wherein the CDR1-L, CDR2-L and CDR3-L comprised in the light chain variable region are the same as the CDR1-L, CDR2-L and CDR3-L as comprised in SEQ ID NO: 60; wherein the CDR1-H, CDR2-H and CDR3-H comprised in the heavy chain variable region are the same as the CDR1-H, CDR2-H and CDR3-H as comprised in SEQ ID NO: 59. In an embodiment, CDR1-L comprised in the light chain variable region is as set forth in SEQ ID NO: 56, CDR2-L is as set forth in SEQ ID NO: 57, and CDR3-L is as set forth in SEQ ID NO: 58; CDR1-H comprised in the heavy chain variable region is as set forth in SEQ ID NO: 53, CDR2-H is as set forth in SEQ ID NO: 54, and CDR3-H is as set forth in SEQ ID NO: 55. In an embodiment, the PD1-targeting antibody comprises a light chain variable region and a heavy chain variable region, wherein the light chain variable region has at least 90%, at least 91%, at least 92%, at least 93%, at least 94%, at least 95%, at least 96%, at least 97%, at least 98%, at least 99%, or 100% identity to SEQ ID NO: 60; wherein the heavy chain variable region has at least 90%, at least 91%, at least 92%, at least 93%, at least 94%, at least 95%, at least 96%, at least 97%, at least 98%, at least 99%, or 100% identity to SEQ ID NO: 59. In a preferred embodiment, the anti-PD1 antibody of the present disclosure comprises a heavy chain variable region as set forth in SEQ ID NO: 59 and a light chain variable region as set forth in SEQ ID NO:60. In an embodiment, the PD1-targeting antibody has at least 90%, at least 91%, at least 92%, at least 93%, at least 94%, at least 95%, at least 96%, at least 97%, at least 98%, at least 99%, or 100% identity to SEQ ID NO: 61. Preferably, the PD1-targeting antibody is as set forth in SEQ ID NO: 61.

In an embodiment, the immunosuppressive protein-binding domain comprised in the immunosuppressive molecule of the present disclosure is the extracellular region of a PD1 ligand, for example, the extracellular regions of PDL1 and PDL2. In this embodiment, the immunosuppressive molecule may further comprise the corresponding transmembrane region of PDL1 or PDL2, but generally does not comprise the corresponding intracellular region thereof at the same time. That is, the immunosuppressive molecule is not full-length PDL1 or PDL2. In an embodiment, the extracellular region of PDL1 has at least 90%, at least 91%, at least 92%, at least 93%, at least 94%, at least 95%, at least 96%, at least 97%, at least 98%, at least 99%, or 100% identity to SEQ ID NO: 64; the extracellular region of PDL2 has at least 90%, at least 91%, at least 92%, at least 93%, at least 94%, at least 95%, at least 96%, at least 97%, at least 98%, at least 99%, or 100% identity to SEQ ID NO: 67. The transmembrane region of PDL1 has at least 90%, at least 91%, at least 92%, at least 93%, at least 94%, at least 95%, at least 96%, at least 97%, at least 98%, at least 99%, or 100% identity to SEQ ID NO: 63; the transmembrane region of PDL2 has at least 90%, at least 91%, at least 92%, at least 93%, at least 94%, at least 95%, at least 96%, at least 97%, at least 98%, at least 99%, or 100% identity to SEQ ID NO: 66. In this embodiment, the immunosuppressive molecule may further comprise a signal peptide of PDL1 or a signal peptide of PDL2. The PDL1 signal peptide has at least 90%, at least 91%, at least 92%, at least 93%, at least 94%, at least 95%, at least 96%, at least 97%, at least 98%, at least 99%, or 100% identity to SEQ ID NO: 62; the PDL2 signal peptide has at least 90%, at least 91%, at least 92%, at least 93%, at least 94%, at least 95%, at least 96%, at least 97%, at least 98%, at least 99%, or 100% identity to SEQ ID NO: 65.

In an embodiment, the immunosuppressive protein-binding domain comprised in the immunosuppressive molecule of the present disclosure is a NKG2A-targeting antibody. Anti-NKG2A antibodies known in the art can all be used in the present disclosure. In an embodiment, the NKG2A-targeting antibody comprises a light chain variable region and a heavy chain variable region, wherein the CDR1-L, CDR2-L and CDR3-L comprised in the light chain variable region are the same as the CDR1-L, CDR2-L and CDR3-L as comprised in SEQ ID NO: 49; wherein the CDR1-H, CDR2-H and CDR3-H comprised in the heavy chain variable region are the same as the CDR1-H, CDR2-H and CDR3-H as comprised in SEQ ID NO: 48. In an embodiment, CDR1-L comprised in the light chain variable region is as set forth in SEQ ID NO: 45, CDR2-L is as set forth in SEQ ID NO: 46, and CDR3-L is as set forth in SEQ ID NO: 47; CDR1-H comprised in the heavy chain variable region is as set forth in SEQ ID NO: 42, CDR2-H is as set forth in SEQ ID NO: 43, and CDR3-H is as set forth in SEQ ID NO: 44. In an embodiment, the NKG2A-targeting antibody comprises a light chain variable region and a heavy chain variable region, wherein the light chain variable region has at least 90%, at least 91%, at least 92%, at least 93%, at least 94%, at least 95%, at least 96%, at least 97%, at least 98%, at least 99%, or 100% identity to SEQ ID NO: 49; wherein the heavy chain variable region has at least 90%, at least 91%, at least 92%, at least 93%, at least 94%, at least 95%, at least 96%, at least 97%, at least 98%, at least 99%, or 100% identity to SEQ ID NO: 48. In a preferred embodiment, the anti-NKG2A antibody of the present disclosure comprises a heavy chain variable region as set forth in SEQ ID NO: 48 and a light chain variable region as set forth in SEQ ID NO:49. In an embodiment, the NKG2A-targeting antibody has at least 90%, at least 91%, at least 92%, at least 93%, at least 94%, at least 95%, at least 96%, at least 97%, at least 98%, at least 99%, or 100% identity to SEQ ID NO: 50. Preferably, the NKG2A-targeting antibody is as set forth in SEQ ID NO: 50.

In an embodiment, the immunosuppressive protein-binding domain of the present disclosure comprises the extracellular region of an NKG2A ligand, for example, the extracellular region of HLA-E. In an embodiment, the extracellular region of HLA-E has at least 90%, at least 91%, at least 92%, at least 93%, at least 94%, at least 95%, at least 96%, at least 97%, at least 98%, at least 99%, or 100% identity to SEQ ID NO: 51 or 52. In this embodiment, the immunosuppressive molecule may further comprise the corresponding transmembrane region of HLA-E, but generally does not comprise the corresponding intracellular region thereof at the same time. That is, the immunosuppressive molecule is not full-length HLA-E.

In an embodiment, the immunosuppressive protein-binding domain of the present disclosure is a FasL-targeting antibody. Anti-FasL antibodies known in the art can all be used in the present disclosure. In an embodiment, the FasL-targeting antibody comprises a light chain variable region and a heavy chain variable region, wherein the CDR1-L, CDR2-L and CDR3-L comprised in the light chain variable region are the same as the CDR1-L, CDR2-L and CDR3-L as comprised in SEQ ID NO: 75; wherein the CDR1-H, CDR2-H and CDR3-H comprised in the heavy chain variable region are the same as the CDR1-H, CDR2-H and CDR3-H as comprised in SEQ ID NO: 74. In an embodiment, CDR1-L comprised in the light chain variable region is as set forth in SEQ ID NO: 71, CDR2-L is as set forth in SEQ ID NO: 72, and CDR3-L is as set forth in SEQ ID NO: 73; CDR1-H comprised in the heavy chain variable region is as set forth in SEQ ID NO: 68, CDR2-H is as set forth in SEQ ID NO: 69, and CDR3-H is as set forth in SEQ ID NO: 70. In an embodiment, the FasL-targeting antibody comprises a light chain variable region and a heavy chain variable region, wherein the light chain variable region has at least 90%, at least 91%, at least 92%, at least 93%, at least 94%, at least 95%, at least 96%, at least 97%, at least 98%, at least 99%, or 100% identity to SEQ ID NO: 75; wherein the heavy chain variable region has at least 90%, at least 91%, at least 92%, at least 93%, at least 94%, at least 95%, at least 96%, at least 97%, at least 98%, at least 99%, or 100% identity to SEQ ID NO: 74. In a preferred embodiment, the anti-FasL antibody of the present disclosure comprises a heavy chain variable region as set forth in SEQ ID NO: 74 and a light chain variable region as set forth in SEQ ID NO:75. In an embodiment, the FasL-targeting antibody has at least 90%, at least 91%, at least 92%, at least 93%, at least 94%, at least 95%, at least 96%, at least 97%, at least 98%, at least 99%, or 100% identity to SEQ ID NO: 76. Preferably, the FasL-targeting antibody is as set forth in SEQ ID NO: 76.

In an embodiment, the immunosuppressive protein-binding domain of the present disclosure comprises the extracellular region of a FasL ligand, for example, the extracellular region of Fas. In an embodiment, the extracellular region of Fas has at least 90%, at least 91%, at least 92%, at least 93%, at least 94%, at least 95%, at least 96%, at least 97%, at least 98%, at least 99%, or 100% identity to SEQ ID NO: 77. In this embodiment, the immunosuppressive molecule may further comprise the corresponding transmembrane region of Fas, but generally does not comprise the corresponding intracellular region thereof at the same time. That is, the immunosuppressive molecule is not full-length Fas. In an embodiment, the transmembrane region of Fas has at least 90%, at least 91%, at least 92%, at least 93%, at least 94%, at least 95%, at least 96%, at least 97%, at least 98%, at least 99%, or 100% identity to SEQ ID NO: 78.

In an embodiment, the immunosuppressive protein-binding domain of the present disclosure is a CTLA4-targeting antibody. Anti-CTLA4 antibodies known in the art can all be used in the present disclosure. In an embodiment, the CTLA4-targeting antibody comprises a light chain variable region and a heavy chain variable region, wherein the CDR1-L, CDR2-L and CDR3-L comprised in the light chain variable region are the same as the CDR1-L, CDR2-L and CDR3-L as comprised in SEQ ID NO: 86; wherein the CDR1-H, CDR2-H and CDR3-H comprised in the heavy chain variable region are the same as the CDR1-H, CDR2-H and CDR3-H as comprised in SEQ ID NO: 85. In an embodiment, CDR1-L comprised in the light chain variable region is as set forth in SEQ ID NO: 82, CDR2-L is as set forth in SEQ ID NO: 83, and CDR3-L is as set forth in SEQ ID NO: 84; CDR1-H comprised in the heavy chain variable region is in SEQ ID NO: 79, CDR2-H is as set forth in SEQ ID NO: 80, and CDR3-H is as set forth in SEQ ID NO: 81; or CDR1-H is as set forth in SEQ ID NO: 10, CDR2-H is as set forth in SEQ ID NO: 11, and CDR3-H is as set forth in SEQ ID NO: 12. In an embodiment, the CTLA4-targeting antibody comprises a light chain variable region and a heavy chain variable region, wherein the light chain variable region has at least 90%, at least 91%, at least 92%, at least 93%, at least 94%, at least 95%, at least 96%, at least 97%, at least 98%, at least 99%, or 100% identity to SEQ ID NO: 86; wherein the heavy chain variable region has at least 90%, at least 91%, at least 92%, at least 93%, at least 94%, at least 95%, at least 96%, at least 97%, at least 98%, at least 99%, or 100% identity to SEQ ID NO: 85. In a preferred embodiment, the anti-CTLA4 antibody of the present disclosure comprises a heavy chain variable region as set forth in SEQ ID NO: 85 and a light chain variable region as set forth in SEQ ID NO:86. In an embodiment, the CTLA4-targeting antibody has at least 90%, at least 91%, at least 92%, at least 93%, at least 94%, at least 95%, at least 96%, at least 97%, at least 98%, at least 99%, or 100% identity to SEQ ID NO: 87. Preferably, the CTLA4-targeting antibody is as set forth in SEQ ID NO: 87.

In an embodiment, the immunosuppressive protein-binding domain of the present disclosure comprises the extracellular region of a CTLA4 ligand, for example, the extracellular region of CD80 or CD86. In an embodiment, the extracellular region of CD80 has at least 90%, at least 91%, at least 92%, at least 93%, at least 94%, at least 95%, at least 96%, at least 97%, at least 98%, at least 99%, or 100% identity to SEQ ID NO: 88; the extracellular region of CD86 has at least 90%, at least 91%, at least 92%, at least 93%, at least 94%, at least 95%, at least 96%, at least 97%, at least 98%, at least 99%, or 100% identity to SEQ ID NO: 89. In this embodiment, the immunosuppressive molecule may further comprise the corresponding transmembrane region of CD80 or CD86, but generally does not comprise the corresponding intracellular region thereof at the same time. That is, the immunosuppressive molecule is not full-length CD80 or CD86. In an embodiment, the transmembrane region of CD80 has at least 90%, at least 91%, at least 92%, at least 93%, at least 94%, at least 95%, at least 96%, at least 97%, at least 98%, at least 99%, or 100% identity to SEQ ID NO: 90. In an embodiment, the transmembrane region of CD86 has at least 90%, at least 91%, at least 92%, at least 93%, at least 94%, at least 95%, at least 96%, at least 97%, at least 98%, at least 99%, or 100% identity to SEQ ID NO: 91.

In an embodiment, the immunosuppressive protein-binding domain of the present disclosure is an SIRPα-targeting antibody. Anti-SIRPα antibodies known in the art can all be used in the present disclosure. In an embodiment, the SIRPα-targeting antibody comprises a light chain variable region and a heavy chain variable region, wherein the CDR1-L, CDR2-L and CDR3-L comprised in the light chain variable region are the same as the CDR1-L, CDR2-L and CDR3-L as comprised in SEQ ID NO: 99; wherein the CDR1-H, CDR2-H and CDR3-H comprised in the heavy chain variable region are the same as the CDR1-H, CDR2-H and CDR3-H as comprised in SEQ ID NO: 98. In an embodiment, CDR1-L comprised in the light chain variable region is as set forth in SEQ ID NO: 95, CDR2-L is as set forth in SEQ ID NO: 96, and CDR3-L is as set forth in SEQ ID NO: 97; CDR1-H comprised in the heavy chain variable region is as set forth in SEQ ID NO: 92, CDR2-H is as set forth in SEQ ID NO: 93, and CDR3-H is as set forth in SEQ ID NO: 94. In an embodiment, the SIRPα-targeting antibody comprises a light chain variable region and a heavy chain variable region, wherein the light chain variable region has at least 90%, at least 91%, at least 92%, at least 93%, at least 94%, at least 95%, at least 96%, at least 97%, at least 98%, at least 99%, or 100% identity to SEQ ID NO: 99; wherein the heavy chain variable region has at least 90%, at least 91%, at least 92%, at least 93%, at least 94%, at least 95%, at least 96%, at least 97%, at least 98%, at least 99%, or 100% identity to SEQ ID NO: 98. In a preferred embodiment, the anti-SIRPα antibody of the present disclosure comprises a heavy chain variable region as set forth in SEQ ID NO: 98 and a light chain variable region as set forth in SEQ ID NO:99. In an embodiment, the SIRPα-targeting antibody has at least 90%, at least 91%, at least 92%, at least 93%, at least 94%, at least 95%, at least 96%, at least 97%, at least 98%, at least 99%, or 100% identity to SEQ ID NO: 100. Preferably, the SIRPα-targeting antibody is as set forth in SEQ ID NO: 100.

In an embodiment, the immunosuppressive protein-binding domain of the present disclosure comprises the extracellular region of an SIRPα ligand, for example, the extracellular region of CD47. In this embodiment, the immunosuppressive molecule may further comprise the corresponding transmembrane region of CD47, or even comprise a segment of its intracellular region, but generally does not comprise a whole intracellular region at the same time. That is, the immunosuppressive molecule is not full-length CD47. In an embodiment, the immunosuppressive molecule comprises an amino acid sequence having at least 90%, at least 91%, at least 92%, at least 93%, at least 94%, at least 95%, at least 96%, at least 97%, at least 98%, at least 99% or 100% identity to SEQ ID NOs: 110, 111, 112, 113, 114, 115, 116, 117, 118 or 119.

In an embodiment, the immunosuppressive protein-binding domain of the present disclosure is a IRP60-targeting antibody. Anti-IRP60 antibodies known in the art can all be used in the present disclosure. In an embodiment, the IRP60-targeting antibody comprises a light chain variable region and a heavy chain variable region, wherein the CDR1-L, CDR2-L and CDR3-L comprised in the light chain variable region are the same as the CDR1-L, CDR2-L and CDR3-L as comprised in SEQ ID NO: 108; wherein the CDR1-H, CDR2-H and CDR3-H comprised in the heavy chain variable region are the same as the CDR1-H, CDR2-H and CDR3-H as comprised in SEQ ID NO: 107. In an embodiment, CDR1-L comprised in the light chain variable region is as set forth in SEQ ID NO: 104, CDR2-L is as set forth in SEQ ID NO: 105, and CDR3-L is as set forth in SEQ ID NO: 106; CDR1-H comprised in the heavy chain variable region is as set forth in SEQ ID NO: 101, CDR2-H is as set forth in SEQ ID NO: 102, and CDR3-H is as set forth in SEQ ID NO: 103. In an embodiment, the IRP60-targeting antibody comprises a light chain variable region and a heavy chain variable region, wherein the light chain variable region has at least 90%, at least 91%, at least 92%, at least 93%, at least 94%, at least 95%, at least 96%, at least 97%, at least 98%, at least 99%, or 100% identity to SEQ ID NO: 108; wherein the heavy chain variable region has at least 90%, at least 91%, at least 92%, at least 93%, at least 94%, at least 95%, at least 96%, at least 97%, at least 98%, at least 99%, or 100% identity to SEQ ID NO: 107. In a preferred embodiment, the anti-IRP60 antibody of the present disclosure comprises a heavy chain variable region as set forth in SEQ ID NO: 107 and a light chain variable region as set forth in SEQ ID NO:108. In an embodiment, the IRP60-targeting antibody has at least 90%, at least 91%, at least 92%, at least 93%, at least 94%, at least 95%, at least 96%, at least 97%, at least 98%, at least 99%, or 100% identity to SEQ ID NO: 109. Preferably, the IRP60-targeting antibody is as set forth in SEQ ID NO: 109.

In an embodiment, the engineered cell expresses an exogenous immunosuppressive molecule, which comprises one or more binding domains that specifically bind to the following immunosuppressive proteins: PD1, NKG2A, FasL, CTLA4, IRP60, and SIRPα. In an embodiment, the binding domain is an antibody targeting the immunosuppressive protein or an extracellular region of a ligand of the immunosuppressive protein. For example, the immunosuppressive molecule comprises an anti-PD1 antibody, an anti-NKG2A antibody, an anti-FasL antibody, an anti-CTLA4 antibody, an anti-SIRPα antibody, an anti-IRP60 antibody, an extracellular region of Fas, an extracellular region of CD80, an extracellular region of CD86, an extracellular region of PDL1, an extracellular region of PDL2, an extracellular region of HLA-E, an extracellular region of CD47, or a combination thereof, for example, the combination of an anti-NKG2A antibody (or an extracellular region of HLA-E) and an anti-PD1 antibody (or an extracellular region of PDL1, an extracellular region of PDL2), an anti-NKG2A antibody (or an extracellular region of HLA-E) and an anti-FasL antibody (or an extracellular region of Fas), an anti-NKG2A antibody (or an extracellular region of HLA-E) and an anti-CTLA4 antibody (or an extracellular region of CD80, an extracellular region of CD86), an anti-NKG2A antibody (or an extracellular region of HLA-E) and an anti-SIRPα antibody (or an extracellular region of CD47), an anti-NKG2A antibody (or an extracellular region of HLA-E) and an anti-IRP60 antibody, an anti-PD1 antibody (or an extracellular region of PDL1, an extracellular region of PDL2) and an anti-FasL antibody (or an extracellular region of Fas), an anti-PD1 antibody (or an extracellular region of PDL1, an extracellular region of PDL2) and an anti-CTLA4 antibody (or an extracellular region of CD80, an extracellular region of CD86), an anti-PD1 antibody (or an extracellular region of PDL1, an extracellular region of PDL2) and an anti-SIRPα antibody (or an extracellular region of CD47), an anti-PD1 antibody (or an extracellular region of PDL1, an extracellular region of PDL2) and an anti-IRP60 antibody, an anti-FasL antibody (or an extracellular region of Fas) and an anti-CTLA4 antibody (or an extracellular region of CD80, an extracellular region of CD86), an anti-FasL antibody (or an extracellular region of Fas) and an anti-SIRPα antibody (or an extracellular region of CD47), an anti-FasL antibody (or an extracellular region of Fas) and an anti-IRP60 antibody, an anti-CTLA4 antibody (or an extracellular region of CD80, an extracellular region of CD86) and an anti-SIRPα antibody (or an extracellular region of CD47), an anti-CTLA4 antibody (or an extracellular region of CD80, an extracellular region of CD86) and an anti-IRP60 antibody, or an anti-SIRPα antibody (or an extracellular region of CD47) and an anti-IRP60 antibody (the two binding domains in the combination may be linked by a linker).

In an embodiment, the engineered cell expresses two or more exogenous immunosuppressive molecules, which comprise a binding domain that binds a different immunosuppressive protein selected from the group consisting of: PD1, NKG2A, FasL, CTLA4, IRP60, and SIRPα, respectively. For example, the two exogenous immunosuppressive molecules bind NKG2A and PD1, NKG2A and CTLA4, NKG2A and FasL, NKG2A and SIRPα, NKG2A and IRP60, PD1 and CTLA4, PD1 and FasL, PD1 and SIRPα, PD1 and IRP60, CTLA4 PD1 and SIRPα, CTLA4 and IRP60, FasL and SIRPα, FasL and IRP60, SIRPα and IRP60, or CTLA4 and FasL, respectively. For example, in an embodiment, the engineered cell expresses two exogenous immunosuppressive molecules, which comprise an anti-NKG2A antibody (or an extracellular region of HLA-E) and an anti-PD1 antibody (or an extracellular region of PDL1, an extracellular region of PDL2), an anti-NKG2A antibody (or an extracellular region of HLA-E) and an anti-FasL antibody (or an extracellular region of Fas), an anti-NKG2A antibody (or an extracellular region of HLA-E) and an anti-CTLA4 antibody (or an extracellular region of CD80, an extracellular region of CD86), an anti-NKG2A antibody (or an extracellular region of HLA-E) and an anti-SIRPα antibody (or an extracellular region of CD47), an anti-NKG2A antibody (or an extracellular region of HLA-E) and an anti-IRP60 antibody, an anti-PD1 antibody (or an extracellular region of PDL1, an extracellular region of PDL2) and an anti-FasL antibody (or an extracellular region of Fas), an anti-PD1 antibody (or an extracellular region of PDL1, an extracellular region of PDL2) and an anti-CTLA4 antibody (or an extracellular region of CD80, an extracellular region of CD86), an anti-PD1 antibody (or an extracellular region of PDL1, an extracellular region of PDL2) and an anti-SIRPα antibody (or an extracellular region of CD47), an anti-PD1 antibody (or an extracellular region of PDL1, an extracellular region of PDL2) and an anti-IRP60 antibody, an anti-FasL antibody (or an extracellular region of Fas) and an anti-CTLA4 antibody (or an extracellular region of CD80, an extracellular region of CD86), an anti-FasL antibody (or an extracellular region of Fas) and an anti-SIRPα antibody (or an extracellular region of CD47), an anti-FasL antibody (or an extracellular region of Fas) and an anti-IRP60 antibody, an anti-CTLA4 antibody (or an extracellular region of CD80, an extracellular region of CD86) and an anti-SIRPα antibody (or an extracellular region of CD47), an anti-CTLA4 antibody (or an extracellular region of CD80, an extracellular region of CD86) and an anti-IRP60 antibody, or an anti-SIRPα antibody (or an extracellular region of CD47) and an anti-IRP60 antibody, respectively. In this embodiment, the two exogenous immunosuppressive molecules can be linked by a 2A peptide (e.g., F2A, T2A, etc.). As used herein, “A (or B) and C (or D, E)” encompasses various combinations of A and C, A and D, A and E, B and C, B and D, and B and E, etc.

As used herein, the term “transmembrane domain” refers to a polypeptide structure that enables the expression of an immunosuppressive molecule on the cell surface, and anchors the immunosuppressive protein-binding domain to the cell membrane. The transmembrane domain may be natural or synthetic, and also may be derived from any membrane-bound protein or transmembrane protein. The transmembrane domain is capable of signaling when a target binding domain binds to a target. The transmembrane domain particularly suitable for the present disclosure can be derived from, for example, a TCR α chain, a TCR β chain, a TCR γ chain, a TCR δ chain, a CD3 ζ subunit, a CD3 ε subunit, a CD3 γ subunit, a CD3 δ subunit, CD28, CD45, CD4, CD5, CD8a, CD9, CD16, CD22, CD33, CD37, CD47, CD64, CD80, CD86, CD94, CD134, CD137, CD154, KIRDS2, OX40, CD2, CD27, CD18, ICOS, 4-1BB, GITR, CD40, BAFFR, HVEM, SLAMF7, NKp80, CD160, BCMA, IL-2R β, IL-2R γ, IL-7R a, ITGA1, VLA1, CD49a, ITGA4, IA4, CD49D, ITGA6, VLA-6, CD49f, ITGAD, CDl 1d, ITGAE, CD103, ITGAL, CDl 1a, LFA-1, ITGAM, CDl 1b, ITGAX, CDl 1c, ITGB1, CD29, ITGB2, CD18, LFA-1, ITGB7, TNFR2, DNAM1, SLAMF4, CD84, CD96, CEACAM1, CRT AM, Ly9, CD160, PSGL1, CDIOO, SLAMF6, SLAMF1, SLAMF8, CD162, LTBR, PAG/Cbp, NKp44, NKp30, NKp46, NKG2D or NKG2C. In some embodiments, the transmembrane domain is derived from the following molecules: CD8a, CD4, CD28, 4-1BB, CD47, CD80, CD86, CD152 and PD1. Alternatively, the transmembrane domain may be synthetic and may mainly comprise hydrophobic residues such as leucine and valine. Preferably, the transmembrane domain is derived from CD28, which has at least 70%, preferably at least 80%, more preferably at least 90%, 95%, 97%, or 99%, or 100% sequence identity to the amino acid sequence as set forth in SEQ ID NO: 14. Preferably, the transmembrane domain is derived from CD8a, which has at least 70%, preferably at least 80%, more preferably at least 90%, 95%, 97%, or 99%, or 100% sequence identity to the amino acid sequence as set forth in SEQ ID NO: 15 or 16.

In an embodiment, the immunosuppressive molecule further comprises a hinge region located between the immunosuppressive protein-binding domain and the transmembrane domain. As used herein, the term “hinge region” generally refers to any oligopeptide or polypeptide that functions to link a transmembrane domain to an antibody. Specifically, the hinge region serves to provide greater flexibility and accessibility to the antibody. The hinge region may comprise up to 300 amino acids, preferably 10 to 100 amino acids and most preferably 25 to 50 amino acids. The hinge region may be completely or partially derived from a natural molecule, e.g., completely or partially from the extracellular region of CD8, CD4 or CD28, or completely or partially from an antibody constant region. Alternatively, the hinge region may be a synthetic sequence corresponding to a naturally existing hinge sequence, or may be a completely synthetic hinge sequence. In a preferred embodiment, the hinge region comprises a hinge region moiety of CD8α, CD28, an FcγRIIIα receptor, IgG4 or IgG1, more preferably a hinge from CD8α, CD28 or IgG4. In an embodiment, the hinge region is from CD28, which has at least 70%, preferably at least 80%, more preferably at least 90%, 95%, 97%, or 99%, or 100% sequence identity to the amino acid sequence as set forth in SEQ ID NO: 26. In an embodiment, the hinge region is from CD8a, which has at least 70%, preferably at least 80%, more preferably at least 90%, 95%, 97%, or 99%, or 100% sequence identity to the amino acid sequence as set forth in SEQ ID NO: 27 or 28. In an embodiment, the hinge region is from IgG4, which has at least 70%, preferably at least 80%, more preferably at least 90%, 95%, 97%, or 99%, or 100% sequence identity to the amino acid sequence as set forth in SEQ ID NO: 29.

As used herein, a “co-stimulatory domain” refers to at least a moiety of a protein that mediates intracellular signal transduction to induce an immune reaction such as an effector function, which is an intracellular functional signaling domain from a co-stimulatory molecule, comprising the entire intracellular region of the co-stimulatory molecule, or a functional fragment thereof. A “co-stimulatory molecule” refers to a cognate binding partner that specifically binds to a co-stimulatory ligand, thereby mediating a co-stimulatory response (e.g., proliferation and survival). The co-stimulatory signaling domain of any co-stimulatory molecule is suitable for the immunosuppressive molecule as described herein. The co-stimulatory molecule includes, but is not limited to, MHC class 1 molecules, BTLA, and Toll ligand receptors. Non-limiting examples of the co-stimulatory domain of the present disclosure include, but are not limited to, intracellular regions derived from the following proteins: LTB, CD94, TLR1, TLR2, TLR3, TLR4, TLR5, TLR6, TLR7, TLR8, TLR9, TLR10, CARD11, CD2, CD7, CD8, CD18, CD27, CD28, CD30, CD40, CD54, CD83, CD134, 4-1BB, CD270, CD272, B7-H3, ICOS, CD357, DAP10, DAP12, LAT, NKG2C, SLP76, PD-1, LIGHT, TRIM, ZAP70 and a combination thereof. Preferably, the co-stimulatory domain of the CAR of the present disclosure is 4-1BB, CD28 or 4-1BB+CD28. In an embodiment, the co-stimulatory domain is from 4-1BB, which has at least 70%, preferably at least 80%, more preferably at least 90%, 95%, 97%, or 99%, or 100% sequence identity to the amino acid sequence as set forth in SEQ ID NO: 18 or 19. In an embodiment, the co-stimulatory domain is from CD28, which has at least 70%, preferably at least 80%, more preferably at least 90%, 95%, 97%, or 99%, or 100% sequence identity to the amino acid sequence as set forth in SEQ ID NO: 17.

The immunosuppressive molecule of the present disclosure does not comprise a primary signaling domain. As used herein, the term “primary signaling domain” refers to a protein structure that acts together after antigen-receptor binding to trigger primary signaling, which is generally an intracellular sequence of a T cell receptor and a co-receptor. The primary signaling domain generally comprises one or more Immunoreceptor Tyrosine-based Activation Motifs (ITAM). Non-limiting examples of the primary signaling domain of the present disclosure include, but are not limited to, those derived from FcRγ, FcRβ, CD3γ, CD3δ, CD3ε, CD3ζ, CD5, CD22, CD79a, CD79b, NFAM1, STAM1, STAM2, and CD66d. In an embodiment, the immunosuppressive molecule of the present disclosure does not comprise a CD35 intracellular region. For example, the Cd3ζ intracellular region has at least 70%, preferably at least 80%, more preferably at least 90%, 95%, 97%, or 99%, or 100% sequence identity to the amino acid sequence as set forth in SEQ ID NO: 21, 22 or 20.

In an embodiment, the immunosuppressive molecule of the present disclosure further comprises a signal peptide such that when it is expressed in a cell, e.g. a T cell, the nascent protein is directed to the endoplasmic reticulum and subsequently to the cell surface. The core of the signal peptide may contain a long hydrophobic amino acid segment, which has a tendency to form a single α-helix. At the end of the signal peptide, there is usually an amino acid segment recognized and cleaved by a signal peptidase. The signal peptidase may cleave during or after translocation, so as to generate a free signal peptide and a mature protein. Then, the free signal peptide is digested by a specific protease. The signal peptides that may be used in the present disclosure is well known to those skilled in the art, for example, the signal peptide derived from B2M, CD8α, IgG1, GM-CSFRα, etc. In an embodiment, the signal peptide that can be used in the present disclosure is from B2M, which has at least 70%, preferably at least 80%, more preferably at least 90%, 95%, 97%, or 99%, or 100% sequence identity to the amino acid sequence as set forth in SEQ ID NO: 23. In an embodiment, the signal peptide that can be used in the present disclosure is from CD8a, which has at least 70%, preferably at least 80%, more preferably at least 90%, 95%, 97%, or 99%, or 100% sequence identity to the amino acid sequence as set forth in SEQ ID NO: 24 or 25.

### III. Functional Exogenous Receptors

In an embodiment, the engineered cell of the present disclosure can further express a functional exogenous receptor. Preferably, the functional exogenous receptor is selected from a recombinant T cell receptor, a chimeric antigen receptor, a T cell fusion protein or T cell antigen coupler, more preferably a chimeric antigen receptor.

As used herein, the term “T cell fusion protein” or “TFP” refers to a recombinant polypeptide derived from each component of a TCR, usually composed of a TCR subunit and an antibody linked thereto, and expressed on the cell surface. Wherein, the TCR subunit includes at least part of the TCR extracellular domain, the transmembrane domain, and the TCR intracellular signal domain.

As used herein, the term “T cell antigen coupler” or “TAC” includes three functional domains: 1 a tumor targeting domain, including a single-chain antibody, a designed ankyrin repeat protein (DARPin) or other targeting groups; 2 an extracellular region domain, a CD3-binding single-chain antibody, so that TAC receptors and TCR receptors are close to each other; 3 a transmembrane region and the intracellular region of a CD4 co-receptor, wherein the intracellular region is linked to protein kinase LCK, to catalyze the phosphorylation of the immunoreceptor tyrosine activation motif (ITAM) of the TCR complex as an initial step in T cell activation.

As used herein, the term “T cell receptor” or “TCR” is a characteristic marker on the surface of T cells that binds to CD3 with a non-covalent bond to form a complex. Antigen-presenting cells present antigenic peptides to T cells through major histocompatibility complex molecules (MHC) and bind to TCR complexes to induce a series of intracellular signaling. TCR is composed of six peptide chains that form heterodimers, respectively, which are generally divided into αβ type and γδ type. Each peptide chain includes a constant region and a variable region, wherein the variable region is responsible for binding specific antigen and MHC molecules. The term “recombinant TCR receptor” refers to an artificially constructed T cell receptor that further comprises an antigen (e.g. a tumor antigen) binding domain.

As used herein, the term “chimeric antigen receptor” or “CAR” refers to an artificially constructed hybrid polypeptide that generally includes an extracellular domain (e.g. an antibody or a ligand of an antigen, or a functional fragment thereof) that specifically binds an antigen (e.g. a tumor antigen), a transmembrane domain, an optional co-stimulatory domain, and an primary signaling domain, and each domain is linked by a linker. CARs are able to reposition the specificity and reactivity of T cells and other immune cells to a selected target in a non-MHC-restricted manner. In an embodiment, the functional exogenous receptor of the present disclosure is a chimeric antigen receptor, which comprises a tumor antigen binding domain, a transmembrane domain, one or more co-stimulatory domains, and a primary signaling domain. In an embodiment, the chimeric antigen receptor further comprises one or more of the following structures: a signal peptide, a hinge region, a suicide gene, a switch structure, etc.

Various linker sequences known in the art can all be used in the present disclosure to link each domain contained in the functional exogenous receptor or immunosuppressive molecule. Examples of linkers include, but are not limited to, (GS) linkers, for example, (GS)n, (GSGGS)n, (GGGS)n, (GGGGS)n, where n is an integer greater than 1. In an embodiment, the linker sequence is as set forth in SEQ ID NO: 31. In an embodiment, the linker sequence is as set forth in SEQ ID NO: 32.

In an embodiment, the functional exogenous receptor comprises an extracellular domain that specifically recognizes an antigen (e.g. a tumor antigen). In an embodiment, the extracellular domain comprises an antibody that specifically binds an antigen or a ligand of the antigen, or a functional fragment of the antigen ligand (i.e., an antigen binding region). In an embodiment, the antigen is selected from the group consisting of: ALK, ADRB3, AKAP-4, APRIL, ASGPR1, BCMA, B7H3, B7H4, B7H6, bcr-abl, BORIS, BST2, BAFF-R, BTLA, CD2, CD3, CD4, CD5, CD7, CD8, CD19, CD20, CD22, CD24, CD25, CD28, CD30, CD33, CD38, CD40, CD44, CD44v6, CD44v7/8, CD47, CD52, CD56, CD57, CD58, CD70, CD72, CD79a, CD79b, CD80, CD81, CD86, CD97, CD123, CD133, CD137, CD 138, CD151, CD171, CD179a, CD300LF, CLEC12A, CDH16, CSPG4, CS1, CLL-1, Claudin 6, Claudin18.1, Claudin 18.2, CEA, CEACAM6, c-Met, CAIX, CXORF61, CA125, CYP1B1, CS1, ELF2M, EGFR, EPCAM, EGFRVIII, EphA2, ERG/TMPRSS2ETS fusion gene, ETV6-AML, EMR2, EGP2, EGP40, FAP, FAR, FBP, FLT3, FOSL1, FCRL5, FCAR, Flt3, Flt4, Frizzled, GD2, GD3, gp100, gp130, GM3, GPC2, GPC3, GPRC5D, GPR20, GloboH, GHRHR, GHR, GITR, Her2, HER3, HER-4, HMWMAA, HAVCR1, HPV E6,E7, HVEM, HIV-1Gag, HLA-A1, HLA-A2, IL6R, IL-11Ra, IL-13Ra, IGF-I receptor, LTPR, LIFRP, LRP5, IGLL1, IGFIR, KIT, Kappa Light Chain, KDR, LewisY, LMP2, LY6K, LAGE-1a, legumain, LCK, LAIR1, LILRA2, LY75, MSLN, MUC1, MUC16, MAGE-A1, MAGE3, MAD-CT-1, MelanA/MART1, ML-IAP, MYCN, mut hsp70-2, NCAM, NY-BR-1, NY-ESO-1, NA17, Notch-1-4, nAchR, NKG2D, NKG2D ligand, OY-TES1, OR51E2, OX40, PRSS21, PSCA, PD1, PD-L1, PD-L2, PSMA, Prostase, PAP, PDGFR-β, PCTA-1/galectin 8, p53, p53 mutant, prostein, PLAC1, PANX3, PAX3, PAX5, PTCH1, RANK, RAGE-1, ROR1, Ras mutant, RhoC, RU1, RU2, Robol, SSEA-4, SSX2, SART3, Sp17, TSHR, Tn Ag, TGS5, TEM1/CD248, TEM7R, TARP, TCRα, TCRβ, TGFBR1, TGFBR2, TNFRSF4, TWEAK-R, TLR7, TLR9, TAG72, TROP-2, Tie 2, TRP-2, TNFR1, TNFR2, TEM1, UPK2VEGFR, WT1, XAGE1, 5T4, 8H9, αvβ6 integrin, CA9, folate receptor a, ephrin B2, tyrosinase, fucosyl GM1, o-acetyl-GD2, folate receptor β, polysialic acid, sperm protein 17, survivin and telomerase, sarcoma translocation breakpoint, human telomerase reverse transcriptase/hTERT, androgen receptor, intestinal carboxylesterase, cyclin B1, fibronectin, tenascin, carcinoembryonic variant of tumor necrosis region, or any combination thereof. Preferably, the antigen is selected from CD7, CD19, CD20, CD22, CD30, CD33, CD38, CD123, CD138, CD171, MUC1, MSLN, AFP, folate receptor a, CEA, PSCA, PSMA, Her2, EGFR, IL-13Ra, GD2, NKG2D, Claudin18.2, ROR1, EGFRVIII, CS1, BCMA and GPRC5D, more preferably selected from CD19, Claudin18.2, MSLN, GPRC5D, CD7 and BCMA.

In an embodiment, the functional exogenous receptor comprises an extracellular domain that specifically recognizes CD19, for example, a CD19-targeting antibody. Anti-CD19 antibodies known in the art can all be used in the present disclosure. In an embodiment, the CD19-targeting antibody comprises a light chain variable region and a heavy chain variable region, wherein the CDR1-L, CDR2-L and CDR3-L comprised in the light chain variable region are the same as the CDR1-L, CDR2-L and CDR3-L as comprised in SEQ ID NO: 12; wherein the CDR1-H, CDR2-H and CDR3-H comprised in the heavy chain variable region are the same as the CDR1-H, CDR2-H and CDR3-H as comprised in SEQ ID NO: 11. In an embodiment, CDR1-L comprised in the light chain variable region is as set forth in SEQ ID NO: 8, CDR2-L is as set forth in SEQ ID NO: 9, and CDR3-L is as set forth in SEQ ID NO: 10; CDR1-H comprised in the heavy chain variable region is as set forth in SEQ ID NO: 5, CDR2-H is as set forth in SEQ ID NO: 6, and CDR3-H is as set forth in SEQ ID NO: 7. In an embodiment, the CD19-targeting antibody comprises a light chain variable region and a heavy chain variable region, wherein the light chain variable region has at least 90%, at least 91%, at least 92%, at least 93%, at least 94%, at least 95%, at least 96%, at least 97%, at least 98%, at least 99%, or 100% identity to SEQ ID NO: 12; wherein the heavy chain variable region has at least 90%, at least 91%, at least 92%, at least 93%, at least 94%, at least 95%, at least 96%, at least 97%, at least 98%, at least 99%, or 100% identity to SEQ ID NO: 11. In a preferred embodiment, the anti-CD19 antibody of the present disclosure comprises a heavy chain variable region as set forth in SEQ ID NO: 11 and a light chain variable region as set forth in SEQ ID NO:12. In an embodiment, the CD19-targeting antibody has at least 90%, at least 91%, at least 92%, at least 93%, at least 94%, at least 95%, at least 96%, at least 97%, at least 98%, at least 99%, or 100% identity to SEQ ID NO: 13. Preferably, the CD19-targeting antibody is as set forth in SEQ ID NO: 13.

In an embodiment, the functional exogenous receptor comprises an extracellular domain that specifically recognizes Claudin18.2, for example, a Claudin18.2-targeting antibody. Anti-Claudin18.2 antibodies known in the art can all be used in the present disclosure. In an embodiment, the Claudin18.2-targeting antibody is a single domain antibody, and the CDR1, CDR2 and CDR3 comprised therein are the same as the CDR1, CDR2 and CDR3 as comprised in SEQ ID NO: 4. In an embodiment, CDR1 is as set forth in SEQ ID NO: 1, CDR2 is as set forth in SEQ ID NO: 2, and CDR3 is as set forth in SEQ ID NO: 3. In an embodiment, the Claudin18.2-targeting antibody has at least 90%, at least 91%, at least 92%, at least 93%, at least 94%, at least 95%, at least 96%, at least 97%, at least 98%, at least 99%, or 100% identity to SEQ ID NO: 4. Preferably, the SIRPα-targeting antibody is as set forth in SEQ ID NO: 4.

In an embodiment, the functional exogenous receptor comprises an extracellular domain that specifically recognizes ROR1, for example, a ROR1-targeting antibody. Anti-ROR1 antibodies known in the art can all be used in the present disclosure. In an embodiment, the ROR1-targeting antibody comprises a light chain variable region and a heavy chain variable region, wherein the CDR1-L, CDR2-L and CDR3-L comprised in the light chain variable region are the same as the CDR1-L, CDR2-L and CDR3-L as comprised in SEQ ID NO: 40; wherein the CDR1-H, CDR2-H and CDR3-H comprised in the heavy chain variable region are the same as the CDR1-H, CDR2-H and CDR3-H as comprised in SEQ ID NO: 39. In an embodiment, CDR1-L comprised in the light chain variable region is as set forth in SEQ ID NO: 36, CDR2-L is as set forth in SEQ ID NO: 37, and CDR3-L is as set forth in SEQ ID NO: 38; CDR1-H comprised in the heavy chain variable region is as set forth in SEQ ID NO: 33, CDR2-H is as set forth in SEQ ID NO: 34, and CDR3-H is as set forth in SEQ ID NO: 35. In an embodiment, the ROR1-targeting antibody comprises a light chain variable region and a heavy chain variable region, wherein the light chain variable region has at least 90%, at least 91%, at least 92%, at least 93%, at least 94%, at least 95%, at least 96%, at least 97%, at least 98%, at least 99%, or 100% identity to SEQ ID NO: 40; wherein the heavy chain variable region has at least 90%, at least 91%, at least 92%, at least 93%, at least 94%, at least 95%, at least 96%, at least 97%, at least 98%, at least 99%, or 100% identity to SEQ ID NO: 39. In a preferred embodiment, the anti-ROR1 antibody of the present disclosure comprises a heavy chain variable region as set forth in SEQ ID NO: 39 and a light chain variable region as set forth in SEQ ID NO:40. In an embodiment, the ROR1-targeting antibody has at least 90%, at least 91%, at least 92%, at least 93%, at least 94%, at least 95%, at least 96%, at least 97%, at least 98%, at least 99%, or 100% identity to SEQ ID NO: 41. Preferably, the ROR1-targeting antibody is as set forth in SEQ ID NO: 41.

In an embodiment, the functional exogenous receptor of the present disclosure is a chimeric antigen receptor, which comprises an antigen binding domain, a transmembrane domain, one or more co-stimulatory domains, and a primary signaling domain. In an embodiment, the functional exogenous receptor of the present disclosure is a chimeric antigen receptor targeting CD19, CD7, CD19, CD20, CD22, CD30, CD33, CD38, CD123, CD138, CD171, MUC1, MSLN, AFP, folate receptor a, CEA, PSCA, PSMA, Her2, EGFR, IL-13Ra, GD2, NKG2D, Claudin18.2, ROR1, EGFRVIII, CS1, BCMA or GPRC5D, more preferably targeting CD19, Claudin18.2, MSLN, ROR1, GPRC5D, CD7 and BCMA. In an embodiment, the chimeric antigen receptor further comprises a signal peptide, a hinge region, or both. For the definitions of the transmembrane domain, co-stimulatory domain, primary signaling domain, and the optional hinge region, signal peptide and other structures that can be used in the CAR of the present disclosure, see the above “immunosuppressive molecules” section. In an embodiment, the transmembrane domain comprised in the CAR may be the same as or different from the transmembrane domain comprised in the immunosuppressive molecule of the present disclosure. In an embodiment, the co-stimulatory domain comprised in the CAR may be the same as or different from the co-stimulatory domain comprised in the immunosuppressive molecule of the present disclosure.

In an embodiment, the CAR of the present disclosure may further comprise a switch structure to regulate the expression time of the CAR. For example, the switch structure may be in the form of a dimerization domain, which undergoes a conformational change by binding to its corresponding ligand, exposing the extracellular binding domain, allowing it to bind to the targeted antigen, thereby activating the signal transduction pathway. Alternatively, switch domains may also be used to link the binding domain and the signaling domain, respectively, and only when the switch domains are coupled with each other (e.g., in the presence of an inducing compound), the binding domain and the signaling domain can be linked together through the dimerization, thereby activating the signaling pathway. The switch structure may further be in the form of a masking peptide. The masking peptide can mask the extracellular binding region and prevent it from binding to the targeted antigen. When the masking peptide is cleaved by, for example, protease, the extracellular binding region can be exposed, making it a “normal” CAR structure. Various switch structures known to those skilled in the art can all be used in the present disclosure.

In an embodiment, the CAR of the present disclosure may further comprise a suicide gene, i.e., to make it express a cell death signal that can be induced by an exogenous substance, so as to eliminate CAR cells when needed (e.g., when severe toxic side effects occur). For example, the suicide gene may be in the form of inserted epitopes, for example, CD20 epitopes, RQR8, etc., and when needed, CAR cells can be eliminated by adding antibodies or reagents targeting these epitopes. The suicide gene may also be herpes simplex virus thymidine kinase (HSV-TK), which causes cell death induced by ganciclovir treatment. The suicide gene may further be iCaspase-9, and the dimerization of iCaspase-9 can be induced by a chemically inducing medicament, e.g., AP1903, AP20187, etc., thereby activating the downstream Caspase3 molecule and leading to apoptosis. Various suicide genes known to those skilled in the art can all be used in the present disclosure.

### IV. Engineered Cells

In an embodiment, the engineered cell of the present disclosure is an engineered immune cell.

As used herein, the term “immune cell” refers to any cell of the immune system that has one or more effector functions (e.g., cytotoxic cell killing activity, secretion of cytokines, induction of ADCC and/or CDC). For example, the immune cell may be a B cell, a T cell, a macrophage, a dendritic cell, a monocyte, an NK cell, or an NKT cell. The immune cell may be obtained from a variety of sources, for example from a subject (e.g., isolated from peripheral blood monocytes, bone marrow, lymph node tissue, cord blood, thymus tissue, tissue from sites of infection, ascites, pleural effusion, spleen tissue, tumors, etc. of the subject), or from a cell line cultured in vitro (e.g., Jurkat, SupT1, NK92, etc.), or differentiated from stem cells (e.g., derived from cord blood stem cells, progenitor cells, bone marrow stem cells, hematopoietic stem cells, adult stem cells, embryonic stem cells, pluripotent stem cells, iPSCs, etc.). Preferably, the immune cell is a T cell or an NK cell, more preferably a T cell. The T cell also may be concentrated or purified. The T cell may be at any stage of development including, but not limited to, a CD4+CD8+ T cell, a CD4+ T cell (e.g., Th1 and Th2 cells), a CD8+ T cell (e.g., a cytotoxic T cell), a CD4-CD8-T cell, a tumor infiltrating cell, a memory T cell, a naive T cell, a γδ-T cell, an αβ-T cell, etc. In a preferred embodiment, the immune cell is a human T cell. The T cell may be isolated from the blood of a subject using a variety of techniques known to those of skill in the art, e.g., Ficoll.

In an embodiment, the engineered cell of the present disclosure is an engineered stem cell. Examples of stem cell include, but are not limited to, a cord blood stem cell, a progenitor cell, a bone marrow stem cell, a hematopoietic stem cell, an adult stem cell, an embryonic stem cell, a pluripotent stem cell, an iPSC, etc.

In an embodiment, the engineered cell of the present disclosure is an allogeneic cell. As used herein, the term “allogeneic” refers to any material derived from a different animal or a different patient of the same species as the individual into whom the material is introduced. Two or more individuals are considered allogeneic to each other when the genes at one or more loci differ. In some cases, allogeneic material from individuals of the same species may be genetically different enough for antigenic interactions to occur.

### V. Pharmaceutical Compositions

The present disclosure further provides a pharmaceutical composition, which comprises the engineered cell of the present disclosure as an active agent, and one or more pharmaceutically acceptable excipients. Therefore, the present disclosure further encompasses use of the engineered cell in the preparation of a pharmaceutical composition or a medicament.

As used herein, the term “pharmaceutically acceptable excipient” refers to a vector and/or excipient that is pharmacologically and/or physiologically compatible (i.e., capable of triggering a desired therapeutic effect without causing any undesired local or systemic effects) with the subject and active ingredient, and it is well known in the art (see, e.g., Remington's Pharmaceutical Sciences. Edited by Gennaro AR, 19th ed. Pennsylvania: Mack Publishing Company, 1995). Examples of pharmaceutically acceptable excipient include, but are not limited to, filler, binder, disintegrant, coating agent, adsorbent, anti-adherent, glidant, antioxidant, flavoring agent, colorant, sweetener, solvent, co-solvent, buffer agent, chelating agent, surfactant, diluent, wetting agent, preservative, emulsifier, cladding agent, isotonic agent, absorption delaying agent, stabilizer, and tension regulator. It is known to those skilled in the art to select a suitable excipient to prepare the desired pharmaceutical composition of the present disclosure. Exemplary excipients for use in the pharmaceutical composition of the present disclosure include saline, buffered saline, dextrose, and water. Generally, the selection of a suitable excipient depends, in particular, on the active agent used, the disease to be treated, and the desired dosage form of the pharmaceutical composition.

The pharmaceutical composition according to the present disclosure is suitable for multiple routes of administration. Generally, the administration is parenterally accomplished. Parenteral delivery methods include topical, intraarterial, intramuscular, subcutaneous, intramedullary, intrathecal, intraventricular, intravenous, intraperitoneal, intrauterine, intravaginal, sublingual, or intranasal administration.

The pharmaceutical composition according to the present disclosure may also be prepared in various forms, e.g., solid, liquid, gaseous or lyophilized forms, and particularly may be in a form of ointment, cream, transdermal patch, gel, powder, tablet, solution, aerosol, granule, pill, suspension, emulsion, capsule, syrup, elixir, extract, tincture or liquid extract, or in a form particularly suitable for the desired method of administration. Processes known in the present disclosure for producing a medicament may include, for example, conventional mixing, dissolving, granulating, sugar-coating, grinding, emulsifying, encapsulating, embedding or lyophilizing process. The pharmaceutical composition comprising, for example, the immune cell as described herein is generally provided in a form of solution, and preferably comprises a pharmaceutically acceptable buffer agent.

The pharmaceutical composition according to the present disclosure may further be administered in combination with one or more other agents (biological formulations such as antibody reagents, and/or small molecules) or treatment methods (e.g., surgery, chemotherapy or radiotherapy) suitable for the treatment and/or prophylaxis of diseases to be treated. Preferred examples of an agent suitable for the combination include known anti-cancer medicaments, e.g., cisplatin, maytansine derivatives, rachelmycin, calicheamicin, docetaxel, etoposide, gemcitabine, ifosfamide, irinotecan, melphalan, mitoxantrone, sorfimer sodiumphotofrin II, temozolomide, topotecan, trimetreate glucuronate, auristatin E, vincristine and doxorubicin; peptide cytotoxins, e.g., ricin, diphtheria toxin, pseudomonas exotoxin A, DNase and RNase; radionuclides, e.g., iodine 131, rhenium 186, indium 111, iridium 90, bismuth 210, bismuth 213, actinides 225 and astatine 213; prodrugs, e.g., antibody-directed enzyme prodrugs; immunostimulatory agents, e.g., platelet factor 4, melanoma growth stimulating protein, etc.; antibodies or fragments thereof, e.g., anti-CD3 antibodies or fragments thereof, complement activators, heterologous protein domains, homologous protein domains, viral/bacterial protein domains and viral/bacterial peptides. In addition, the pharmaceutical composition of the present disclosure can also be used in combination with one or more other treatment methods, for example, chemotherapy and radiotherapy.

In an embodiment, the pharmaceutical composition of the present disclosure is used for the treatment of a subject with cancer, infection or autoimmune diseases.

In an embodiment, the cancer is a cancer associated with the expression of a target binding to a functional exogenous receptor. For example, the cancer includes, but is not limited to, brain glioma, blastoma, sarcoma, leukemia, basal cell carcinoma, biliary tract cancer, bladder cancer, bone cancer, brain and CNS cancer, breast cancer, peritoneal cancer, cervical cancer, choriocarcinoma, colon and rectal cancer, connective tissue cancer, cancer of the digestive system, endometrial cancer, esophageal cancer, eye cancer, head and neck cancer, gastric cancer (including gastrointestinal cancer), glioblastoma (GBM), liver cancer, hepatoma, intraepithelial neoplasia, kidney cancer, laryngeal cancer, liver tumor, lung cancer (e.g., small cell lung cancer, non-small cell lung cancer, adenocarcinoma of the lung, and squamous cell lung cancer), lymphoma (including Hodgkin's lymphoma and non-Hodgkin's lymphoma), melanoma, myeloma, neuroblastoma, oral cancer (e.g., lip, tongue, mouth, and pharynx), ovarian cancer, pancreatic cancer, prostate cancer, retinoblastoma, rhabdomyosarcoma, colorectal cancer, cancer of the respiratory system, salivary gland cancer, skin cancer, squamous cell carcinoma, gastric cancer, testicular cancer, thyroid cancer, uterine or endometrial cancer, malignancy of the urinary system, vulvar cancer, and other cancers and sarcomas, and B-cell lymphoma (including low-grade/follicular non-Hodgkin's lymphoma (NHL), small lymphocytic (SL) NHL, intermediate-grade/follicular NHL, intermediate-grade diffuse NHL, high-grade immunoblastic NHL, high-grade lymphoblastic NHL, high-grade small non-cleaved cell NHL, large mass disease NHL), mantle cell lymphoma, AIDS-related lymphoma, and Waldenstrom macroglobulinemia, chronic lymphocytic leukemia (CLL), acute lymphoblastic leukemia (ALL), B-cell acute lymphoblastic leukemia (B-ALL), T-cell acute lymphoblastic leukemia (T-ALL), B-cell prolymphocytic leukemia, blastic plasmacytoid dendritic cell neoplasm, Burkitt's lymphoma, diffuse large B-cell lymphoma, follicular lymphoma, chronic myeloid leukemia (CML), malignant lymphoproliferative disease, MALT lymphoma, hairy cell leukemia, marginal zone lymphoma, multiple myeloma, myelodysplasia, plasmablastic lymphoma, preleukemia, plasmacytoid dendritic cell neoplasm, and post-transplant lymphoproliferative disorder (PTLD); and other diseases associated with the expression of a target. Preferably, the disease that can be treated with the engineered immune cell or the pharmaceutical composition of the present disclosure is selected from the group consisting of: leukemia, lymphoma, multiple myeloma, brain glioma, pancreatic cancer, gastric cancer, etc.

In an embodiment, the infection includes, but is not limited to, infections caused by viruses, bacteria, fungi, and parasites.

In an embodiment, the autoimmune disease includes, but is not limited to type I diabetes, celiac disease, Graves' disease, inflammatory bowel disease, multiple sclerosis, psoriasis, rheumatoid arthritis, Addison's disease, Sjögren's syndrome, Hashimoto's thyroiditis, myasthenia gravis, vasculitis, pernicious anemia and systemic lupus erythematosus.

The present disclosure will be described in detail below with reference to the accompanying drawings and examples. It should be noted that those skilled in the art should understand that the drawings of the present disclosure and the examples thereof are only for the purpose of illustration, and shall not constitute any limitation to the present disclosure. In the case of no contradiction, the examples in the present disclosure and the features in the examples can be combined with each other.

### BRIEF DESCRIPTION OF DRAWINGS

FIG. 1: Killing activity of CD19 CAR-T cells against target cells.

FIG. 2: Cytokine release levels of CD19 CAR-T cells after co-incubation with target cells.

FIG. 3: Survival curves of tumor-bearing mice treated with CD19 CAR-T cells.

FIG. 4: Killing activity of CD19 CAR-T cells with knockout of HLA-II, Fas, TGFBR1, TGFBR2 or the combination thereof against target cells.

FIG. 5: Cytokine release levels of CD19 CAR-T cells with knockout of HLA-II, Fas, TGFBR1, TGFBR2 or the combination thereof after co-incubation with target cells.

FIG. 6: In vivo tumor suppression effect of CD19 CAR-T cells with knockout of HLA-II, Fas, TGFBR1, TGFBR2 or the combination thereof.

FIG. 7: Killing activity of CLDN18.2 CAR-T cells against target cells.

FIG. 8: Amplification levels of CLDN18.2 CAR-T cells.

FIG. 9: Killing activity of ROR1 CAR-T cells against target cells.

FIG. 10: Amplification levels of ROR1 CAR-T cells.

FIG. 11: Suppressive effects of engineered B cells on the killing activity against NK92 cells.

### DETAILED DESCRIPTION

### Example 1: Structures of Exemplary CARs and Immunosuppressive Molecules Used in the Examples

The CAR structure comprises an extracellular domain, a CD8a hinge region (SEQ ID NO: 27), a CD8a transmembrane region (SEQ ID NO: 15), a 4-1BB co-stimulatory domain (SEQ ID NO: 18), and a CD3ζ intracellular region (SEQ ID NO: 21) linked in sequence. The extracellular domain comprised in the CD19 CAR structure is an anti-CD19 scFv (SEQ ID NO: 13). The extracellular domain comprised in the Claudin18.2 CAR structure is an anti-Claudin18.2 vhh (SEQ ID NO: 4). The extracellular domain comprised in the ROR1 CAR structure is an anti-ROR1 scFv (SEQ ID NO: 44).

The structure of the PD1-targeting immunosuppressive molecule IIM-PDL1 comprises a PDL1 signal peptide (SEQ ID NO: 62), a PDL1 extracellular region (SEQ ID NO: 64), a PDL1 transmembrane region (SEQ ID NO: 63), and a CD28 co-stimulatory domain (SEQ ID NO: 17) linked in sequence. The structure of the PD1-targeting immunosuppressive molecule IIM-PDL2 comprises a PDL2 signal peptide (SEQ ID NO: 65), a PDL2 extracellular region (SEQ ID NO: 67), a PDL2 transmembrane region (SEQ ID NO: 66), and a CD28 co-stimulatory domain (SEQ ID NO: 17) linked in sequence.

The structure of the NKG2A-targeting immunosuppressive molecule IIM-NKG2A comprises an anti-NKG2A scFv (SEQ ID NO: 50), an IgG4 hinge region (SEQ ID NO: 29), a CD28 transmembrane region (SEQ ID NO: 14), and a CD28 co-stimulatory domain (SEQ ID NO: 17) linked in sequence.

### Example 2. Preparation of CD19 CAR-T Cells

The coding sequence of CD19 CAR was synthesized and cloned into a pGEM-T Easy plasmid (Promega). Three ml Opti-MEM (Gibco) was added to a sterile tube to dilute the above plasmid, and then packaging vector psPAX2 (Addgene) and envelope vector pMD2.G (Addgene) were added according to the ratio of plasmid:viral packaging vector:viral envelope vector=4:2:1. Then, 120 μl X-treme GENE HP DNA transfection reagent (Roche) was added, mixed immediately, and incubated at room temperature for 15 min, and then the plasmid/vector/transfection reagent mixture was added dropwise to the culture flask of 293T cells. Viruses were collected at 24 hours and 48 hours, pooled, and ultracentrifuged (25000 g, 4 °C, 2.5 hours) to obtain concentrated CD19 CAR lentiviruses.

T cells were activated with DynaBeads CD3/CD28 CTSTM (Gibco), and were cultured for 1 day at 37 °C and 5% CO₂. On the second day, the concentrated lentiviruses were added, and after 3 days of continuous culture, T cells expressing CD19 CAR (19 T cells) were obtained. Additionally, the above lentiviruses were used to transfect T cells in which CIITA had been knocked out by the CRISPR/Cas9 system, yielding 19-CII KO T cells; or to transfect T cells in which both CIITA and B2M had been knocked out by the CRISPR/Cas9 system, yielding 19-CI+CII KO T cells. Wild-type T cells not transduced with CAR and without genetic modification (NT cells) were used as controls.

### Example 3. Killing Effects of CD19 CAR-T Cells Against Target Cells

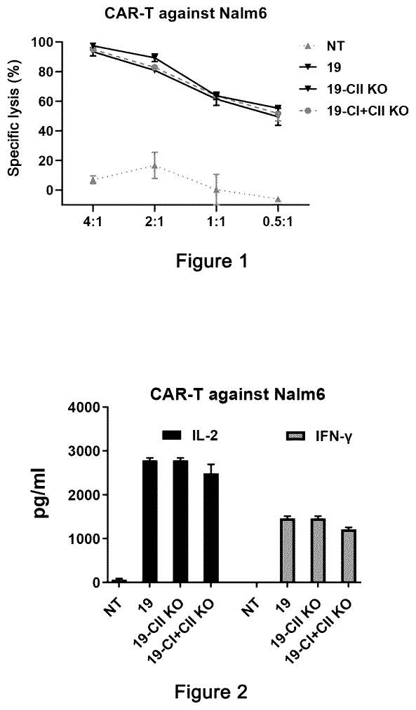

Target cells, Nalm6 cells, were plated into 96-well plates at a concentration of 1×10⁴ cells/well, and then, NT cells, 19 T cells, 19-CII KO T cells and 19-CI+CII KO T cells were plated into 96-well plates at an effector-to-target ratio of 4:1, 2:1, 1:1 or 0.5:1 for co-culture. After 18 hours, the fluorescence value was measured with a microplate reader. According to the calculation formula: (average fluorescence value of target cells-average fluorescence value of samples)/average fluorescence value of target cells×100%, the killing efficiency was calculated, and the results are shown in FIG. 1.

It can be seen that the three kinds of tested CD19-targeting CAR-T cells all showed effective specific killing against the target cells, with comparable killing activity. This indicates that down-regulating HLA-II-related genes or further down-regulating HLA-I-related genes did not adversely affect the in vitro killing activity of the CAR-T cells.

### Example 4. Cytokine Release Levels of CD19 CAR-T Cells after Co-Incubation with Target Cells

Target cells (Nalm6 cells) were plated in 96-well plates at a concentration of 1×10⁵ cells/well, NT cells, 19 T cells, 19-CII KO T cells and 19-CI+CII KO T cells were added at a ratio of 1:1, respectively, and the cell co-culture supernatant was collected after 18-24 hours of co-culture. Human IL-2 DuoSet ELISA Kit (R&D systems) and Human IFN-gamma DuoSet ELISA Kit (R&D systems) were used to detect the contents of IL2 and IFN-γ in the co-culture supernatant, respectively, and the results are shown in FIG. 2. It can be seen that, compared with NT cells, the three types of CAR-T cells showed significantly increased release levels of cytokines IL-2 and IFN-γ after co-culture with target cells, indicating that this cytokine release is specific. In addition, the release levels of IL2 and IFN-γ of the three kinds of CAR-T cells are comparable, indicating that down-regulating HLA-II-related genes or further down-regulating HLA-I-related genes did not affect the cytokine release level of the CAR-T cells.

### Example 5. In Vivo Suppression Effect of CD19 CAR-T Cells on Tumors

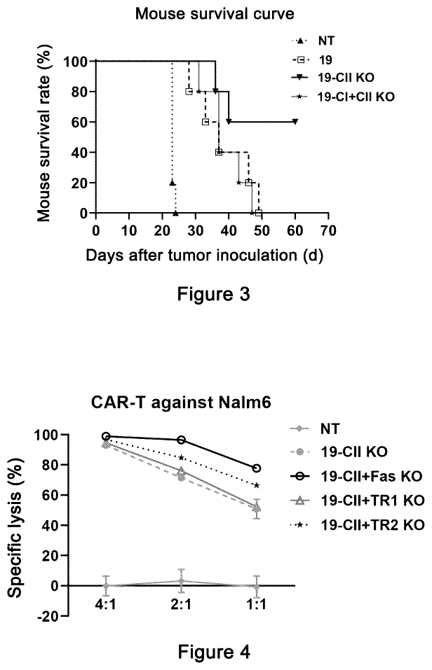

Twenty healthy female NPI mice (a severely immunodeficient mouse model) aged 6-8 weeks were divided into 4 groups, with 5 mice in each group. On day 0, 5×10⁵ Raji cells were injected into the tail vein of each mouse. Six days later, 2×10⁶ NT cells, 19 T cells, 19-CII KO T cells, or 19-CI+CII KO T cells were injected into the tail vein of each mouse according to the grouping. The mice survival status was evaluated weekly, and the mice survival curves are shown in FIG. 3.

The inventors unexpectedly found that the in vivo tumor suppression effect of 19-CI+CII KO T cells was comparable to that of 19 T cells, indicating that simultaneously knocking out HLA-I-related genes and HLA-II-related genes did not compromise the killing activity of CD19 CAR-T cells. However, it was observed that 19-CII KO T cells showed the best tumor suppression effect. This may be attributed to the knocking out HLA-II-related genes enhancing the amplification ability of CAR-T cells, which in turn prolongs the in vivo killing effect time thereof. In short, this indicates that knocking out only HLA-II-related genes can better enhance the efficacy of CAR-T cells compared with simultaneously knocking out HLA-I-related genes and HLA-II-related genes.

### Example 6. Preparation of CD19 CAR-T Cells with Knockout of Multiple Endogenous Genes and Verification of the Function Thereof

CD19 CAR lentiviruses prepared in Example 1 were used to transfect T cells in which CIITA and Fas had been knocked out by the CRISPR/Cas9 system, yielding 19-CII+Fas KO T cells; or to transfect T cells in which CIITA and TGFBR1 had been knocked out by the CRISPR/Cas9 system, yielding 19-CII+TR1 KO T cells; or to transfect T cells in which CIITA and TGFBR2 had been knocked out by the CRISPR/Cas9 system, yielding 19-CII+TR2 KO T cells.

The killing activity of the above CAR-T cells against target cells Nalm6 at effector-to-target ratios of 4:1, 2:1 and 1:1 was detected by the method described in Example 2, and the results are shown in FIG. 4. It can be seen that compared with 19-CII KO T cells, additional knockout of Fas, TGFBR1 or TGFBR2 gene will not negatively impact but may even significantly improve the killing activity of CAR-T cells.

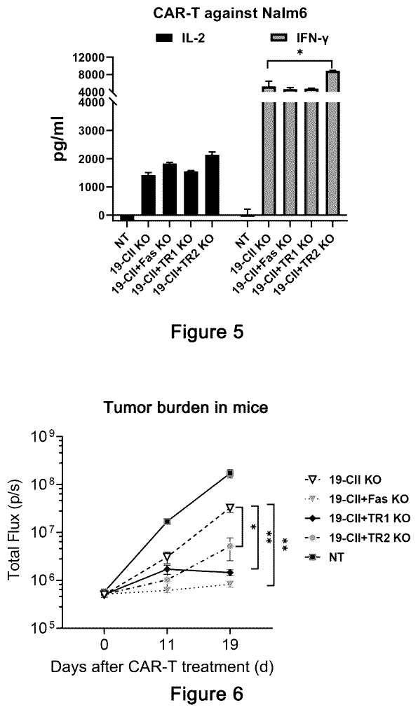

The cytokine release levels of the above CAR-T cells after co-incubation with target cells were detected by the method described in Example 3, and the results are shown in FIG. 5. It can be seen that compared with 19-CII KO T cells, additional knockout of Fas, TGFBR1 genes did not significantly affect the release levels of the cytokines, and knockout of TGFBR2 gene can significantly improve the release level of INF-γ cytokine.

Twenty-five healthy female NPI mice aged 6-8 weeks were divided into 5 groups, with 5 mice in each group. On day 0, 4×10⁶ Raji cells were injected into the tail vein of each mouse. Six days later, 2×10⁶ NT cells, 19-CII KO T cells, 19-CII+Fas KO T cells, 19-CII+TR1 KO T cells, or 19-CII+TR2 KO T cells were injected into the tail vein of each mouse according to the grouping. The status of the mice was assessed weekly. The progression of tumor burden in the mice is shown in FIG. 6. It can be seen that compared with CAR-T cells with knockout of only HLA-II-related genes, further knockout of Fas, TGFBR1 or TGFBR2 gene can significantly enhance the in vivo killing activity of CD19 CAR-T cells.

### Example 7. Preparation of CLDN18.2 CAR-T Cells and Verification of the Function Thereof

The coding sequence of Claudin18.2 CAR was synthesized and cloned into a pGEM-T Easy plasmid (Promega), yielding a CAR18.2 plasmid. The above plasmid was packaged into lentivirus according to the method of Example 1, which was then uses to transfect T cells in which CIITA and B2M had been knocked out by the CRISPR/Cas9 system, yielding 18.2-CI+CII KO T cells.

The above CAR18.2 plasmid further comprised the coding sequence of the PD1-targeting immunosuppressive molecule IIM-PDL1 linked by T2A (SEQ ID NO: 31), yielding the CAR18.2P plasmid, and the correct insertion of the target sequence in the plasmid was confirmed by sequencing. The above plasmid was packaged into lentivirus according to the method of Example 1, which was then uses to transfect T cells in which CIITA and B2M had been knocked out by the CRISPR/Cas9 system, yielding 18.2P-CI+CII KO T cells; or to transfect T cells in which CIITA had been knocked out by the CRISPR/Cas9 system, yielding 18.2P-CII KO T cells.

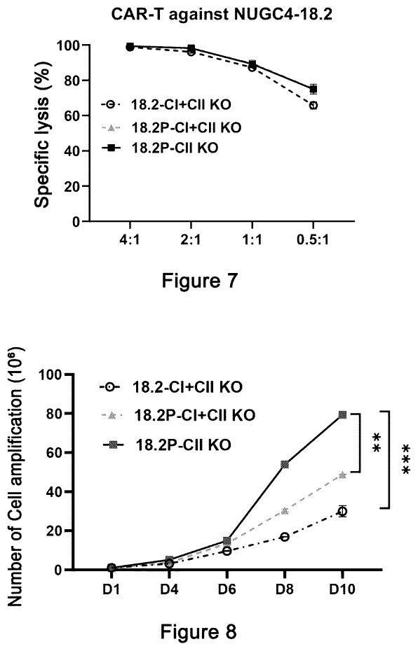

The killing activity of the above CAR-T cells against target cells NUGC4-18.2 was detected by the method of Example 2, and the results are shown in FIG. 7. It can be seen that the killing activities of the three kinds of tested Claudin18.2-targeting CAR-T cells at various effector-to-target ratios are basically comparable.

The proliferation level of the above CAR-T cells was detected by cell counting, and the results are shown in FIG. 8. It can be seen that the proliferation level of 18.2-CI+CII KO T cells is the lowest, followed by 18.2P-CI+CII KO T cells, and the highest is 18.2P-CII KO T cells. This shows that further expressing immunosuppressive molecules in CAR-T cells and replacing HLA-I+HLA-II knockout with HLA-II knockout can significantly improve the proliferation activity of cells.

### Example 8. Preparation of ROR1 CAR-T Cells and Verification of the Function Thereof

The coding sequence of ROR1 CAR was synthesized and cloned into a pGEM-T Easy plasmid (Promega), yielding a ROR1 plasmid. The above plasmid was packaged into lentivirus according to the method of Example 1, which was then uses to transfect T cells in which CIITA and B2M had been knocked out by the CRISPR/Cas9 system, yielding ROR1-CI+CII KO T cells.

The ROR1 plasmid further comprised the coding sequence of the PD1-targeting immunosuppressive molecule IIM-PDL2 and the coding sequence of the NKG2A-targeting immunosuppressive molecule IIM-NKG2A, each linked via T2A, yielding the ROR1PA plasmid, and the correct insertion of the target sequence in the plasmid was confirmed by sequencing. The above plasmid was packaged into lentivirus according to the method of Example 1, which was then uses to transfect T cells in which CIITA and B2M had been knocked out by the CRISPR/Cas9 system, yielding ROR1PA-CI+CII KO T cells; or to transfect T cells in which CIITA had been knocked out by the CRISPR/Cas9 system, yielding ROR1PA-CII KO T cells.

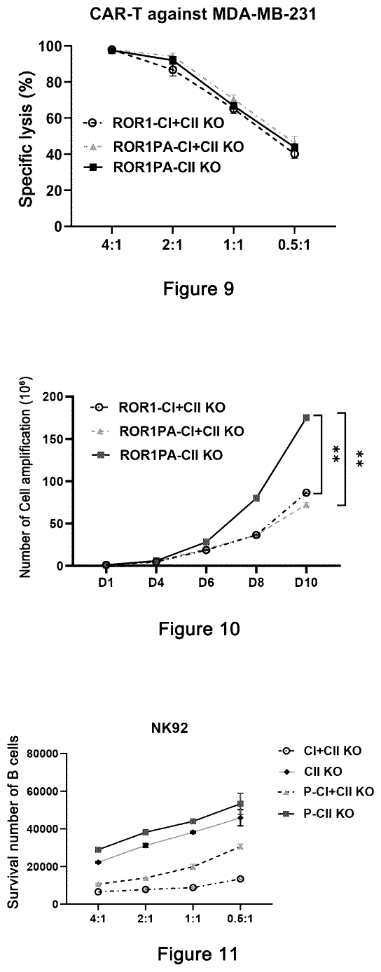

The killing activity of the above CAR-T cells against target cells MDA-MB-231 was detected by the method described in Example 2, and the results are shown in FIG. 9. It can be seen that the killing activities of the three kinds of tested ROR1-targeting CAR-T cells at various effector-to-target ratios are basically comparable.

The proliferation level of the above CAR-T cells was detected by cell counting, and the results are shown in FIG. 10. It can be seen that the proliferation level of ROR1-CI+CII KO T cells is comparable to that of ROR1PA-CI+CII KO T cells, while the proliferation level of ROR1PA-CII KO T cells is the highest. This indicates that simultaneous expression of immunosuppressive molecules and knocking out HLA-II (retaining HLA-I)-related genes in CAR-T cells can significantly improve the proliferation activity of cells.

### Example 9. Preparation of Engineered B Cells and Verification of the Function Thereof

CIITA in B cells was knocked out by the CRISPR/Cas9 system, yielding CII KO B cells; or CIITA and B2M were knocked out, yielding CI+CII KO B cells.

The coding sequence of the PD1-targeting immunosuppressive molecule IIM-PD1 was cloned into a pGEM-T Easy plasmid. The above plasmid was packaged into lentivirus according to the method of Example 1, which was then uses to transfect B cells in which CIITA and B2M had been knocked out by the CRISPR/Cas9 system, yielding P-CI+CII KO B cells; or to transfect B cells in which CIITA had been knocked out by the CRISPR/Cas9 system, yielding P-CII KO B cells.

The suppression effect of the above engineered B cells on the killing activity of NK92 cells was detected. Specifically, the B cells CI+CII KO, CII KO, P-CI+CII KO and P-CII KO prepared by the present disclosure were labeled with Far-Red (invitrogen, Cat. No. C34564). Then, the labeled B cells were plated into 96-well plates according to a concentration of 1×10⁴ cells/well, and NK92 cells were added at effector-to-target ratios of 4:1, 2:1, 1:1 and 0.5:1 for co-culture. After 16-18 hours, the survival number of B cells in the culture was detected by trucount using flow cytometry, and the results are shown in FIG. 11. It can be seen that compared with knocking out HLA-I+HLA-II, knocking out only HLA-II significantly improves the suppression effect of B cells on the killing of NK cells, and the additional expression of immunosuppressive molecules is capable of further enhancing this suppression effect, which in turn improves the survival number of B cells.

In summary, compared with simultaneously knocking out HLA-I and HLA-II, knocking out only HLA-II (i.e., retaining HLA-I) will, on the one hand, not adversely affect the in vitro killing activity or cytokine release level of the engineered cell (e.g. T cell, B cell, etc.) of the present disclosure, but instead enhance their in vivo killing activity (possibly by enhancing the in vivo proliferation ability thereof, which in turn prolongs the duration thereof), thereby enhancing the tumor suppression effect of the engineered cell; on the other hand, it is capable of further suppressing the killing of engineered cells by endogenous immune cells, which in turn reduces the risk of immune rejection. Secondly, the inventors also found that on the basis of knocking out HLA-II (i.e., retaining HLA-I), further knocking out genes such as Fas, TGFBR1, and TGFBR2 can enhance the in vitro and in vivo killing activity of the engineered cell, or additionally expressing immunosuppressive molecules is capable of further suppressing the killing of other immune cells, and ultimately both can achieve a synergistic effect of enhancing the function of the engineered cell.

It should be noted that the above are merely for preferred examples of the present disclosure and not used to limit the present disclosure. For those skilled in the art, various modifications and changes may be made to the present disclosure. Those skilled in the art should understand that any amendments, equivalent replacements, improvements, and so on, made within the spirit and principle of the present disclosure, should be covered within the scope of protection of the present disclosure.

1. An engineered cell, wherein:
(i) expression of at least one of the endogenous HLA-II-related genes is down-regulated; and
(ii) expression of endogenous HLA-I-related genes is not altered.

2. The engineered cell of claim 1, wherein the HLA-II-related gene is selected from the group consisting of: HLA-DP, HLA-DQ, HLA-DR, TAP1, TAP2, LMP2, LMP7, RFX5, RFXAP, RFXANK, CIITA, and a combination thereof.

3. The engineered cell of claim 1, wherein the HLA-I-related gene is HLA-A, HLA-B, HLA-C, and B2M.

4. The engineered cell of claim 1, wherein the expression of at least one of the endogenous TCR/CD3 genes of the engineered cell is down-regulated

5. (canceled)

6. The engineered cell of claim 1, wherein the expression of at least one of the endogenous gene of the engineered cell is down-regulated, the at least one of the endogenous gene selected from the group consisting of: CD52, GR, dCK, PD1, LAG3, TIM3, CTLA4, PPP2CA, PPP2CB, PTPN6, PTPN22, PDCD1, HAVCR2, BTLA, CD160, TIGIT, CD96, CRTAM, TNFR1, DR3, DR4, DR5, TNFRSF10B, TNFRSF10A, CASP8, CASP10, CASP3, CASP6, CASP7, FADD, FAS, TGFBR1, TGFBR2, SMAD2, SMAD3, SMAD4, SMAD10, SKI, SKIL, TGIF1, IL10RA, IL10RB, HMOX2, IL6R, IL6ST, EIF2AK4, CSK, PAG1, SIT, FOXP3, PRDM1, BATF, GUCY1A2, GUCY1A3, GUCY1B2 and GUCY1B3.

7. The engineered cell of claim 1, wherein the engineered cell further expresses an immunosuppressive molecule can reduce the immunogenicity of the engineered cell.

8. The engineered cell of claim 7, wherein the immunosuppressive molecule comprises an immunosuppressive protein-binding domain, a transmembrane domain, and a co-stimulatory domain and does not comprise a primary signaling domain.

9. The engineered cell of claim 8, wherein the immunosuppressive protein-binding domain comprises:
(i) an antibody that specifically binds the following targets: PD1, TIGIT, CD96, TIM3, LAG3, CTLA4, FASL, NKG2A, NKG2B, CD94, LIR1, LIR2, LIR3, LIR5, LIR8, KIR2DL1, KIR2DL2/3, KIR3DL1, CEACAM1, LAIR1, SIGLEC7, SIGLEC9, SIRP-α, IRp60, and KLRG1; and/or
(ii) an extracellular region of a protein selected from the group consisting of: HLA-E, HLA-F, HLA-G, cadherin, collagen, OCIL, sialic acid, PDL1, PDL2, CD155, CD112, CD113, Gal-9, FGL1, FAS, CD80, CD86 and CD47.

10. The engineered cell of claim 9, wherein the immunosuppressive protein-binding domain comprises an anti-PD1 antibody, an anti-NKG2A antibody, an anti-FasL antibody, an anti-CTLA4 antibody, a PDL1 extracellular region, a PDL2 extracellular region, an HLA-E extracellular region, a Fas extracellular region, a CD80 extracellular region, a CD86 extracellular region, or a combination thereof.

11. The engineered cell of claim 8, wherein the transmembrane domain is selected from a transmembrane domain of a protein selected from the group consisting of: a TCR α chain, a TCR β chain, a TCR γ chain, a TCR δ chain, a CD3ζ subunit, a CD3 ε subunit, a CD3 γ subunit, a CD3 δ subunit, CD28, CD45, CD4, CD5, CD8a, CD9, CD16, CD22, CD33, CD37, CD47, CD64, CD80, CD86, CD94, CD134, CD137, CD154, KIRDS2, OX40, CD2, CD27, CD18, ICOS, 4-1BB, GITR, CD40, BAFFR, HVEM, SLAMF7, NKp80, CD160, BCMA, IL-2Rβ, IL-2Rγ, IL-7R a, ITGA1, VLA1, CD49a, ITGA4, IA4, CD49D, ITGA6, VLA-6, CD49f, ITGAD, CDl 1d, ITGAE, CD103, ITGAL, CDl 1a, LFA-1, ITGAM, CDl 1b, ITGAX, CDl 1c, ITGB1, CD29, ITGB2, CD18, LFA-1, ITGB7, TNFR2, DNAM1, SLAMF4, CD84, CD96, CEACAM1, CRT AM, Ly9, CD160, PSGL1, CDIOO, SLAMF6, SLAMF1, SLAMF8, CD162, LTBR, PAG/Cbp, NKp44, NKp30, NKp46, NKG2D or NKG2C.

12. The engineered cell of claim 8, wherein the co-stimulatory domain is selected from a intracellular region of a protein selected from the group consisting of: LTB, CD94, TLR1, TLR2, TLR3, TLR4, TLR5, TLR6, TLR7, TLR8, TLR9, TLR10, CARD11, CD2, CD7, CD8, CD18, CD27, CD28, CD30, CD40, CD54, CD83, CD134, 4-1BB, CD270, CD272, B7-H3, ICOS, CD357, DAP10, DAP12, LAT, NKG2C, SLP76, PD-1, LIGHT, TRIM, ZAP70 and a combination thereof.

13. The engineered cell of claim 8, wherein the immunosuppressive molecule does not comprise a CD3ζ intracellular region.

14. The engineered cell of claim 1, further expressing a functional exogenous receptor, wherein the functional exogenous receptor is selected from the group consisting of: a chimeric antigen receptor, a chimeric T cell receptor, a T cell antigen coupler, and a T cell fusion protein.

15. (canceled)

16. The engineered cell of claim 14, wherein the functional exogenous receptor comprises an extracellular domain that specifically recognizes an antigen.

17. The engineered cell of claim 16, wherein the extracellular domain comprises an antibody that specifically recognizes the antigen or a ligand of the antigen.

18. The engineered cell of claim 16, wherein the antigen is selected from the group consisting of: ALK, ADRB3, AKAP-4, APRIL, ASGPR1, BCMA, B7H3, B7H4, B7H6, bcr-abl, BORIS, BST2, BAFF-R, BTLA, CD2, CD3, CD4, CD5, CD7, CD8, CD19, CD20, CD22, CD24, CD25, CD28, CD30, CD33, CD38, CD40, CD44, CD44v6, CD44v7/8, CD47, CD52, CD56, CD57, CD58, CD69, CD70, CD72, CD79a, CD79b, CD80, CD81, CD86, CD97, CD123, CD133, CD137, CD 138, CD151, CD171, CD179a, CD300LF, CLEC12A, CDH16, CSPG4, CS1, CLL-1, Claudin 6, Claudin18.1, Claudin 18.2, CEA, CEACAM6, c-Met, CAIX, CXORF61, CA125, CYP1B1, CS1, ELF2M, EGFR, EPCAM, EGFRVIII, EphA2, ERG/TMPRSS2ETS fusion gene, ETV6-AML, EMR2, EGP2, EGP40, FAP, FAR, FBP, FLT3, FOSL1, FCRL5, FCAR, Flt3, Flt4, Frizzled, GD2, GD3, gp100, gp130, GM3, GPC2, GPC3, GPRC5D, GPR20, GloboH, GHRHR, GHR, GITR, Her2, HER3, HER-4, HMWMAA, HAVCR1, HPV E6, E7, HVEM, HIV-1Gag, HLA-A1, HLA-A2, IL6R, IL-11Ra, IL-13Ra, IGF-I receptor, LTPR, LIFRP, LRP5, IGLL1, IGF1R, KIT, Kappa Light Chain, KDR, LewisY, LMP2, LY6K, LAGE-1a, legumain, LCK, LAIR1, LILRA2, LY75, MSLN, MUC1, MUC16, MAGE-A1, MAGE3, MAD-CT-1, MelanA/MART1, ML-IAP, MYCN, mut hsp70-2, NCAM, NY-BR-1, NY-ESO-1, NA17, Notch-1-4, nAchR, NKG2D, NKG2D ligand, OY-TES1, OR51E2, OX40, PRSS21, PSCA, PD1, PD-L1, PD-L2, PSMA, Prostase, PAP, PDGFR-β, PCTA-1, galectin 8, p53, p53 mutant, prostein, PLAC1, PANX3, PAX3, PAX5, PTCH1, RANK, RAGE-1, ROR1, Ras mutant, RhoC, RU1, RU2, Robol, SSEA-4, SSX2, SART3, Sp17, TSHR, Tn Ag, TGS5, TEM1/CD248, TEM7R, TARP, TCRα, TCRβ, TNFRSF4, TWEAK-R, TLR7, TLR9, TAG72, TROP-2, Tie 2, TRP-2, TNFR1, TNFR2, TEM1, UPK2VEGFR, WT1, XAGE1, 5T4, 8H9, αvβ6 integrin, CA9, folate receptor α, ephrin B2, tyrosinase, fucosyl GM1, o-acetyl-GD2, folate receptor β, polysialic acid, sperm protein 17, survivin and telomerase, sarcoma translocation breakpoint, human telomerase reverse transcriptase/hTERT, androgen receptor, intestinal carboxylesterase, cyclin B1, fibronectin, tenascin, carcinoembryonic variant of tumor necrosis region, or any combination thereof.

19. (canceled)

20. The engineered cell of claim 1, wherein the engineered cell is a B cell, a T cell, a macrophage, a dendritic cell, a monocyte, an NK cell, or an NKT cell.

21. (canceled)

22. The engineered cell of claim 1, wherein the engineered cell is or differentiated from a stem cell selected from the group consisting of: a cord blood stem cell, a progenitor cell, a bone marrow stem cell, a hematopoietic stem cell, an adult stem cell, an embryonic stem cell, a pluripotent stem cell, and an iPSC.

23. A pharmaceutical composition comprising the engineered cell of claim 1, and one or more pharmaceutically acceptable excipients.

24. A method for treating cancer, infection or autoimmune diseases in a subject, comprising administering the engineered cell of claim 1 to the subject.
